(19) United States
(12) Patent Application Publication
(10) Pub. No.: US 2023/0293306 A1
(43) Pub. Date: Sep. 21, 2023

(54) BIONIC TISSUE STENT, PREPARATION METHOD THEREFOR AND APPLICATION THEREOF

(71) Applicant: SINOBIOPRINT (SHANGHAI) BIOTECH LTD., Shanghai (CN)

(72) Inventors: Harry Huimin CHEN, Shanghai (CN); Hailin ZHU, Shanghai (CN); Zhengguang WANG, Shanghai (CN)

(21) Appl. No.: 18/005,963

(22) PCT Filed: Aug. 6, 2021
(86) PCT No.: PCT/CN2021/111119

§ 371 Date: Jan. 18, 2023

(30) Foreign Application Priority Data
Aug. 6, 2020 (CN) 202010785145.2
Aug. 6, 2020 (CN) 202010785153.7
Aug. 6, 2020 (CN) 202010785161.1

(51) Int. Cl. A61F 2/30; A61L 27/52; A61L 27/16

(52) U.S. Cl. CPC A61F 2/30; A61L 27/52; A61L 27/16

(57) ABSTRACT

Disclosed are a bionic tissue stent, a preparation method therefor and an application thereof. The preparation method of the present invention is a 3D printing demolding method, and the preparation method can achieve the high-precision 3D engineering fabrication of a hydrogel material. The bionic tissue stent obtained by using said preparation method has a microscopic fine mesh structure, pores communicate, porosity is high, and the specific surface area is large, especially for bionic cartilage stents and osteochondral stents; moreover, the present invention has great application value in the treatment of articular cartilage diseases.

The present application claims priority to Chinese Patent Application CN2020107851452 filed on Aug. 6, 2020, Chinese Patent Application CN2020107851611 filed on Aug. 6, 2020 and Chinese Patent Application CN2020107851537 filed on Aug. 6, 2020, the contents of which are incorporated herein by reference herein in their entireties.

### TECHNICAL FIELD

The present invention relates to a bionic tissue scaffold, a preparation method therefor and an application thereof.

### BACKGROUND

As a fundamental pathological process, articular cartilage diseases are involved in the early stages of pathological changes in almost all common clinical joint diseases. Due to the lack of blood vessels and lymphatic distribution in cartilage tissue, the low content of chondrocytes, the lack of grandmother cells necessary for cell differentiation and being embedded in thick extracellular matrix, it is difficult to migrate and cannot effectively move to the site of injury to participate in repair, so its self-repair ability is very poor and even small cartilage defects are difficult to repair naturally. The emergence of tissue engineering technology provides a new idea and method for the treatment of articular cartilage injuries.

Scaffold material is one of the three main elements of tissue engineering technology. There have been many studies on the preparation of bionic cartilage scaffolds using different biomaterials. However, the existing high polymer material bionic cartilage scaffolds are poorly biocompatible during use and the absorption time is difficult to control; while pure natural biomaterial bionic cartilage scaffolds are too low in strength and difficult to prepare; the existing cartilage repair formulas are prone to ossification or fibrosis during use, and there is no clinical product with good cartilage repair effect.

At present, the design of tissue-engineered osteochondral scaffold is mainly divided into the following categories: 1) bone uses scaffolds while cartilage does not use scaffolds, i.e., high-density chondrocytes are directly grown on the bone scaffold; 2) two scaffold materials suitable for bone and cartilage construction are used to form tissue-engineered bone and cartilage respectively by in vitro culture, and then the tissue-engineered bone and cartilage are partially assembled into tissue-engineered osteochondral complex by means of bonding, or surgical suturing, or sequential implantation; 3) both bone and cartilage use an integrated single-layer scaffold of the same scaffold material; 4) bone and cartilage use an integrated bi-layer scaffold of two different scaffold materials respectively.

Bi-layer osteochondral scaffold has better properties because its layered structure is designed according to the needs of bone and cartilage growth. However, biphasic osteochondral scaffold of this type also has the following problems:
1) Poor mechanical properties of the upper cartilage material, water absorption and deformation after implantation, and rapid degradation rate;
2) The repaired cartilage is fibrocartilage rather than hyaline cartilage;
3) Poor integration of the new cartilage and the surrounding cartilage tissue. Established clinical products, which repair osteochondral defects by perfusion, have no through-hole structure, so cells cannot enter the inside of the scaffold and only stay on the surface, resulting in poor repair effect; in addition, they may grow into scar tissues or osteogenesis may occur.
4) The joint is insufficient in mechanical strength and easily separated; it is easy to hinder the penetration of the bone layer and the cartilage layer, leading to cell migration and nutrient delivery obstacles.

3D bio-printing is an emerging technology for constructing tissues and organs, including organoids. The technology has made great strides in recent years, but still has many limitations. One of the most daunting challenges is the accuracy and complexity of bionic tissues. The technical methods of 3D printing comprise inkjet printing, laser-assisted printing or extrusion printing, with extrusion printing being more suitable for 3D bio-printing; wherein extrusion printing is also the most suitable for a wide range of bioinks. However, because the hydrogel material prepared by bioink is too soft and has a long curing time, the accuracy of morphology maintenance decreases and it will collapse, making it difficult to maintain an accurate printing effect.

In general, 3D bio-printing requires a phase transition (photocuring) from a photosensitive hydrogel to a semi-solid crosslinking network via photo-initiated free radical polymerization to form the corresponding biomaterial structure. A better photocuring technology can effectively control/modulate the mechanical properties and degradation rate of the material with good biocompatibility, and can enhance the elasticity of the printed structure and prolong the storage time as needed. However, with the currently used photocuring technology, low-viscosity materials (such as gelatin methacrylate, sodium alginate methacrylate, etc.) are not easily molded, and light intensity and light duration are not easily adjusted precisely, making it difficult to control the hardness and strength of the printed structures and to form fine and complex structures.

### CONTENT OF THE PRESENT INVENTION

The present disclosure provides a preparation method for a bionic tissue scaffold, comprising the following steps of:
S1, carrying out 3D printing with a sacrificial material to obtain a mold; wherein the sacrificial material is a hard high polymer material which can be dissolved in a solvent;

S2, pouring hydrogel composition into the mold for in-situ crosslinking curing, thus obtaining a cured hydrogel-mold complex;

S3, demolding: dissolving the mold in the cured hydrogel-mold complex with a solvent to obtain a cured hydrogel;

S4, freeze-drying the obtained cured hydrogel to obtain the bionic tissue scaffold.

The hard high polymer material of the present disclosure is defined as follows: if the high polymer material is 3D printed with the goal of forming a cube scaffold with a size of 10*10*10 mm, and the actual size error of the scaffold formed is within 10%, the high polymer material can be called a hard high polymer material. The cube scaffold described here is a criterion for judging whether the high polymer material is a hard high polymer material or not, and it does not limit the shape that the material can be formed. 3D printing with hard high polymer materials maintains good morphology and enables high-precision printing.

Conventional sacrificial materials currently commonly used in the art, such as pluronic, carbomer, gelatin particles, sucrose, etc., are unable to maintain their morphology during 3D printing and cannot be finely printed. Hard high polymer materials that do not conform to the present disclosure are, for example, PEEK (polyether ether ketone), PEKK (polyether ketone ketone), PEI (polyetherimide) or PPSU (high-performance medical grade plastics), all of which can be printed by FDM with very good stability, but are not suitable for use as sacrificial materials and cannot be removed under conventional conditions.

The sacrificial material of the present disclosure is preferably biocompatible. The “biocompatible” criterion is that the cell viability is 75% or more when tested for biocompatibility using conventional methods in the art.

The sacrificial material of the present disclosure is preferably transparent or translucent. When the crosslinking curing is a photocrosslinking curing, the sacrificial material must be transparent or translucent.

The sacrificial material of the present disclosure is preferably polylactic acid (PLA), polycaprolactone (PCL), polyethylene terephthalate-1,4-cyclohexanedimethanol ester (PETG), polyvinyl alcohol (PVA) or a synthetic photosensitive resin. The synthetic photosensitive resin is preferably a polyacrylate photosensitive resin.

In step S1 of the present disclosure, preferably, a pigment is first mixed into the sacrificial material, and then the colored sacrificial material is 3D printed to obtain a colored mold. By doing so, the color disappearance can be used as a monitoring indicator for successful removal of the mold in demolding of step S3.

In step S1 of the present disclosure, the method of the 3D printing may be a conventional printing method in the art that can realize precise fine structures, preferably an extrusion method (i.e., a fused deposition method) or a photocuring method. The photocuring method may be stereo lithography apparatus technology (SLA), digital light projection technology (DLP) or liquid crystal display technology (LCD).

In step S1 of the present disclosure, the shape, size and structure of the mold can be designed according to the desired bionic tissue scaffold according to conventional methods in the art.

The hydrogel composition of the present disclosure is a raw material composition for forming a hydrogel, comprising at least a gelable component and a gel medium. The hydrogel composition may be a conventional hydrogel composition used in the art for bionic tissue scaffolds.

In the present disclosure, the components of the hydrogel composition may be present in the form of a mixture, or may be separately dispensed and mixed at the time of use. Wherein, the gel medium is generally not mixed with the gelable component, and when the gel medium is separately dispensed from the gelable component, the gelable component may be in the form of powder, flakes or flocculent.

Wherein, the gelable component may be a component conventional in the art that can be cured to form a gel, generally comprising a natural gelable component and/or a synthetic gelable component.

The natural gelable component may be conventional in the art, preferably comprising one or more selected from a group consisting of natural proteins, natural protein modification products, natural protein degradation products, modification products of natural protein degradation products, natural polysaccharides, natural polysaccharide modification products, natural polysaccharide degradation products and modification products of natural polysaccharide degradation products.

The natural proteins comprise one or more selected from a group consisting of various hydrophilic animal and plant proteins, water-soluble animal and plant proteins, type I collagen, type II collagen, serum proteins, silk fibroin and elastin. The natural protein degradation product preferably includes gelatin (Gel) or polypeptide. The modification product of natural protein degradation product is preferably a natural protein degradation product methacrylate, more preferably gelatin methacrylate (GelMA).

The natural polysaccharide comprises one or more selected from a group consisting of hyaluronic acid (HA), carboxymethyl cellulose, methyl cellulose, hydroxyethyl cellulose, hydroxypropyl cellulose, alginate, dextran, agarose, heparin, chondroitin sulfate (CS), ethylene glycol chitosan, propylene glycol chitosan, chitosan lactate, carboxymethyl chitosan and chitosan quaternary ammonium salt, preferably hyaluronic acid (HA) and/or chondroitin sulfate (CS). The natural polysaccharide modification product is preferably a natural polysaccharide methacrylate, such as hyaluronic acid methacrylate (HAMA) or chondroitin sulfate methacrylate (CSMA).

Herein, the synthetic gelable component may be conventional in the art, preferably comprising one or more selected from a group consisting of two-arm or multi-arm polyethylene glycol diacrylate, polyethyleneimine, synthetic polypeptide, polyacrylic acid, polymethacrylic acid, polyacrylate, polymethacrylate, polyacrylamide, polymethacrylamide, polyvinyl alcohol and polyvinylpyrrolidone.

Preferably, the gelable component comprises one or a combination of more selected from a group consisting of gelatin methacrylate (GelMA), collagen methacrylate, elastin methacrylate, hyaluronic acid methacrylate (HAMA), chondroitin sulfate methacrylate (CSMA), sodium alginate methacrylate, heparin methacrylate, gelatin, collagen, elastin, hyaluronic acid, chondroitin sulfate, heparin and sodium alginate (Alg).

The gelable component of the present disclosure preferably comprises one or more selected from a group consisting of gelatin methacrylate (GelMA), hyaluronic acid methacrylate (HAMA) and chondroitin sulfate methacrylate (CSMA). At this point, the bionic tissue scaffold is generally a cartilage scaffold or a bone scaffold.

In a preferred embodiment of the present disclosure, the bionic tissue scaffold is a cartilage scaffold, and the gelable component comprises sodium alginate (Alg) and gelatin methacrylate (GelMA).

In a preferred embodiment of the present disclosure, the bionic tissue scaffold is a cartilage scaffold, and the gelable component comprises gelatin methacrylate (GelMA) and hyaluronic acid methacrylate (HAMA).

In a preferred embodiment of the present disclosure, the bionic tissue scaffold is a cartilage scaffold, and the gelable component comprises gelatin methacrylate (GelMA), hyaluronic acid methacrylate (HAMA) and chondroitin sulfate methacrylate (CSMA).

In a preferred embodiment of the present disclosure, the bionic tissue scaffold is a cartilage scaffold, and the gelable component comprises sodium alginate (Alg), gelatin methacrylate (GelMA) and hydroxyapatite (HAp).

In a preferred embodiment of the present disclosure, the bionic tissue scaffold is a nerve conduit scaffold, and the gelable component comprises sodium alginate and gelatin methacrylate.

In a preferred embodiment of the present disclosure, the bionic tissue scaffold is a skin scaffold, and the gelable component comprises collagen methacrylate and gelatin methacrylate.

In a preferred embodiment of the present disclosure, the bionic tissue scaffold is a muscle scaffold, and the gelable component comprises hyaluronic acid methacrylate and gelatin methacrylate.

The gel medium of the present disclosure may be conventional in the art, preferably one or more selected from a group consisting of purified water, saline, cell culture medium, calcium salt solution and phosphate buffered solution (PBS solution). Wherein, the saline is 0.9% NaCl aqueous solution. The cell culture medium may be a conventional cell culture medium in the art, such as DMEM, DMEM/F12, RPMI 1640 and other commonly used medium. The phosphate buffered solution may be conventional in the art, the pH of the phosphate buffered solution is preferably 7.4.

The gelatin methacrylate of the present disclosure may be conventional in the art, commercially available, or may be obtained by methacrylation of gelatin (Gel) according to conventional methods in the art.

The methacrylation degree of the gelatin methacrylate may be 30%-100%, preferably 40%-80%. Wherein, the methacrylation degree of the gelatin methacrylate is calculated using hydrogen nuclear magnetic resonance (¹H NMR) by selecting the integrated area of the standard peak of phenylalanine (7.1-7.4 ppm) as 1 and calculating the percentage decrease in peak area of the lysine signal at 2.8-2.95 ppm before and after gelatin modification, i.e., Methacrylation degree of GelMA=(peak area of Gel's lysine signal at 2.8-2.95 ppm−peak area of GelMA's lysine signal at 2.8-2.95 ppm)/peak area of Gel's lysine signal at 2.8-2.95 ppm*100%.

The hyaluronic acid methacrylate of the present disclosure may be conventional in the art, commercially available, or may be obtained by methacrylation of hyaluronic acid (HA) according to conventional methods in the art. The molecular weight of the hyaluronic acid methacrylate may be 1-2000 kDa, preferably 100-1000 kDa, more preferably 500-950 kDa, more preferably 890-950 kDa.

The methacrylation degree of the hyaluronic acid methacrylate may be 20%-60%, preferably 30%-50%. Wherein, the methacrylation degree of the hyaluronic acid methacrylate is calculated using hydrogen nuclear magnetic resonance (¹H NMR) as follows: Methacrylation degree of HAMA=peak area of methacrylate-vinyl at 5.6 ppm/peak area of N-acetyl glucosamine at 1.9 ppm*100%.

The chondroitin sulfate methacrylate of the present disclosure may be conventional in the art, commercially available, or may be obtained by methacrylation of chondroitin sulfate (CS) according to conventional methods in the art. The molecular weight of the chondroitin sulfate methacrylate may be 10-70 kDa, preferably 30-50 kDa.

The methacrylation degree of the chondroitin sulfate methacrylate may be 20%-60%, preferably 30%-50%. Wherein, the methacrylation degree of the chondroitin sulfate methacrylate is calculated using hydrogen nuclear magnetic resonance (¹H NMR) as follows: Methacrylation degree of CSMA=peak area of methacrylate-vinyl at 5.6 ppm/peak area of N-acetyl glucosamine at 1.9 ppm*100%.

When the gelable component of the present disclosure is a photosensitive gelable component, the hydrogel composition further comprises a photoinitiator. When the hydrogel composition comprises a photoinitiator, at the time of use, the photosensitive gelable component is first mixed with a gel medium, and then the photoinitiator is added. The purpose of doing so is that the gelable component is first dissolved in the gel medium to facilitate stable preservation of the hydrogel composition, and the photoinitiator is temporarily added at the time of use to avoid a certain degree of crosslinking of the hydrogel composition due to the presence of the photoinitiator during preservation.

The photoinitiator of the present disclosure may be a conventional photoinitiator in the art, preferably a blue photoinitiator, a ultraviolet photoinitiator or a green photoinitiator; the blue photoinitiator is preferably lithium phenyl-2,4,6-trimethylbenzoylphosphonate (LAP), riboflavin, flavin mononucleotide, eosin Y or terpyridyl ruthenium chloride/sodium persulfate (Ru/SPS); the ultraviolet photoinitiator is preferably 2-hydroxy-2-methyl-1-[4-(2-hydroxyethoxy)phenyl]-1-propanone (I2959).

Herein, the hydrogel composition may further comprise a thickener. The thickener may be conventional in the art, preferably one or more selected from a group consisting of polyethylene oxide (PEO), polyethylene glycol (PEG), sodium alginate (Alg), hyaluronic acid, polyvinylpyrrolidone, gum arabic, gellan gum and xanthan gum.

Herein, the hydrogel composition may further comprise a synthetic photosensitive material. The synthetic photosensitive material may be conventional in the art, preferably comprising one or more selected from a group consisting of polyethylene glycol acrylate (PEGDA), polyacrylic acid, polymethacrylic acid, polyacrylate, polymethacrylate, polyacrylamide and polymethacrylamide. The synthetic photosensitive material is preferably polyethylene glycol acrylate.

The crosslinking curing of the present disclosure may comprise one or more selected from a group consisting of physical crosslinking curing, chemical crosslinking curing and photocrosslinking curing; preferably including photocrosslinking curing. The crosslinking curing may be performed by conventional methods in the art according to the properties of the gelable component.

Herein, the physical crosslinking curing may be performed by conventional methods in the art, such as self-assembly curing of collagen at about 37° C.

Herein, the chemical crosslinking curing may be performed by conventional methods in the art, for example, methacrylated materials are catalyzed by ammonium persulfate to form gels, or, sodium alginate are crosslinked with divalent metal cations to form gels.

Herein, the photocrosslinking curing may be performed under light irradiation by conventional methods in the art; preferably, the photocrosslinking is carried out under light irradiation at a wavelength of 365-405 nm and an intensity of 5-50 mW/cm²; more preferably, the photocrosslinking is carried out under light irradiation at a wavelength of 405 nm and an intensity of 10 mW/cm². When the gelable component is a photosensitive gelable component, the crosslinking curing is a photocrosslinking curing.

In step S3, the solvent may be selected according to the properties of the sacrificial material forming the mold, which only needs to be able to dissolve the mold. The solvent is preferably dichloromethane, trichloromethane, tetrahydrofuran, 1,4-dioxane, purified water, saline, calcium salt solution, phosphate buffered solution (PBS) or culture medium.

In step S4, the freeze-drying is preferably carried out for 8-24 h; the freeze-drying is preferably preceded by a pre-cooling step; the pre-cooling is preferably carried out at a temperature of −20° C., the pre-cooling is preferably carried out for 1-3 h.

The present disclosure also provides a bionic tissue scaffold, which is prepared according to the preparation method for the bionic tissue scaffold.

The bionic tissue scaffold of the present disclosure can bionic the tissues that are conventionally required to be bionic in the art, such as cartilage, bone, nerve conduit, ligament, muscle, breast, fat, skin, heart, liver, spleen, lung, kidney, pancreas, stomach, intestines, bladder, blood vessels, etc.

The preparation method for the bionic tissue scaffold of the present disclosure is an indirect 3D printing method, also known as a 3D printing demolding method or a 3D engineering method, which can achieve the high-precision 3D engineering fabrication of a hydrogel material by selecting a suitable sacrificial material, the obtained bionic tissue scaffold has a microscopic fine mesh structure (penetrating structure), pores communicate, porosity is adjustable (up to 30%-70%), and the specific surface area is large (taking the formation of a scaffold with a side length of 10 mm and a height of 3 mm as an example, the surface area formed by 3D printing through-hole structure (with a filling rate of 50%, a layer height of 0.2 mm and a nozzle size of 0.25 mm) is 3200 mm², while the surface area of the same size scaffold formed by perfusion is 320 mm², with a 10-fold increase in the surface area of the porous scaffold structure compared to that of the non-porous structure).

The present disclosure provides in particular an osteochondral scaffold comprising a cartilage layer, an adhesive layer and a bone layer, the adhesive layer being connected to the cartilage layer and the bone layer on each side; one or more of the cartilage layer, the adhesive layer and the bone layer being porous.

In the present disclosure, preferably, the cartilage layer, the adhesive layer and the bone layer are all porous; more preferably, the pores of the cartilage layer, the pores of the adhesive layer and the pores of the bone layer are communicated. The pores of the cartilage layer, the pores of the adhesive layer and the pores of the bone layer may be completedly or incompletely aligned, preferably completely aligned.

In the present disclosure, the pore diameter of the pores of the cartilage layer and/or the bone layer is 50-350 μm, preferably 200-280 μm, for example 250 μm; preferably, the pore diameter of the pores of the bone layer is equal to that of the pores of the cartilage layer. The pore diameters of the cartilage layer and the bone layer are both selected to be suitable for cell capture and cell growth.

In the present disclosure, the distribution of the pores of the cartilage layer and/or the bone layer is preferably arranged vertically and crosswise.

In the present disclosure, the porosity of the cartilage layer and/or the bone layer is 20%-70%, preferably 40%-60%, for example 50%.

In the present disclosure, the porosity of the osteochondral scaffold may be 20%-70%, preferably 40%-60%, for example 50%.

The adhesive layer of the present disclosure is a transition layer between the cartilage layer and the bone layer, capable of achieving a connection between the cartilage layer and the bone layer, not necessarily connected through bonding.

Preferably, the adhesive layer does not cover or partially covers the pores of the bone layer and/or the cartilage layer. That is, the adhesive layer covers only part or all of the non-porous areas of the bone layer and the cartilage layer to ensure that the adhesive layer does not block the pores of the bone layer and the cartilage layer. The pores of the adhesive layer can be aligned with the pores of the cartilage layer and the pores of the bone layer to ensure a three-layer penetration.

In the present disclosure, the shape of the osteochondral scaffold is not particularly limited, and in use, the osteochondral scaffold can be cut according to the size of the defect site.

For example, the osteochondral scaffold is a cylinder. The cylinder may have a diameter of 2-30 mm, preferably 2-20 mm, more preferably 3-10 mm; the cylinder may have a height of 2-10 mm, more preferably 3-6 mm.

For example, the osteochondral scaffold is a cuboid. The bottom surface of the cuboid can be a square, the square may have a side length of 2-30 mm, preferably 2-20 mm, more preferably 3-10 mm; the cuboid may have a height of preferably 2-10 mm, more preferably 3-6 mm.

In the present disclosure, the height ratio of the bone layer and the cartilage layer may be 1:(0.1-1), preferably 1:(0.2-0.5).

In the present disclosure, the height of the adhesive layer may be from 5 μm to 2 mm, preferably 0.1-2 mm, more preferably 0.5-1 mm.

In the present disclosure, the material of the cartilage layer may be a conventional cartilage layer material in the art, preferably a hydrogel material. Wherein, the hydrogel material may be one or more selected from a group consisting of a single network hydrogel material, an interpenetrating network hydrogel material and a composite crosslinked hydrogel material. A hydrogel material formed by a single crosslinking method is called a single network hydrogel material. A hydrogel material formed by two or more crosslinking methods is called an interpenetrating network hydrogel material, or a double network hydrogel material. A hydrogel material formed by composite crosslinking of various gelable components in the same crosslinking method is called a composite crosslinked hydrogel material. The hydrogel material is preferably a photocrosslinked hydrogel material, more preferably a composite photocrosslinked hydrogel material.

The cartilage layer of the present disclosure is preferably also loaded with a cartilage promoting component. Wherein, the cartilage promoting component may comprise bioactive factors and/or cells. Wherein, the bioactive factors preferably comprise transforming growth factor TGFα or TGFβ. The cells may comprise autologous or allogeneic chondrocytes, mesenchymal stem cells, embryonic stem cells or induced pluripotent stem cells.

In the present disclosure, the material of the bone layer may be a conventional medical high polymer material in the art, preferably polylactic acid (PLA), polylactic-co-glycolic acid (PLGA) or polycaprolactone (PCL).

In the present disclosure, the material of the bone layer may also be a hydrogel material, the hydrogel material being as previously described.

The bone layer of the present disclosure is preferably also loaded with a bone promoting component. Wherein, the bone promoting component may comprise one or more selected from a group consisting of bioactive inorganic materials, bioactive factors and cells.

Herein, the bioactive inorganic material preferably comprises one or more selected from a group consisting of hydroxyapatite, calcium phosphate, calcium carbonate and bioactive glass. The mass percentage of the bioactive inorganic material in the bone layer may be 0.1 wt %-70 wt %, preferably 1 wt %-50 wt %, more preferably 2.5 wt %-30 wt %.

The bioactive factors preferably comprise one or more selected from a group consisting of transforming growth factors TGFα, TGFβ, bone morphogenetic proteins BMP-2, BMP-3, BMP-4, BMP-5, BMP-6, BMP-7, BMP-8 and BMP-9, and cartilage inducing compounds (such as KGN, etc.).

The cells may comprise autologous or allogeneic bone cells, mesenchymal stem cells, embryonic stem cells or induced pluripotent stem cells.

In certain embodiments, the material of the adhesive layer may be a hydrogel material, the hydrogel material being as previously described.

In certain embodiments, the adhesive layer may be formed by a conventional medical glue in the art. The medical glue may be selected from, for example, Compont, Greensea, Golden Elephant, Hynaut, Double One, Domperidone, 3M, 7LK, FuAiLe, IDEALPLAST, Kaiyan or Aofeite.

In the present disclosure, preferably, the cartilage layer, the adhesive layer and the bone layer are all hydrogel materials. At this point, the concentration of the gelable components in the cartilage layer, the adhesive layer and the bone layer varies gradually.

The present disclosure also provides a preparation method for the osteochondral scaffold comprising the following steps of: connecting a bone layer and a cartilage layer, with an adhesive layer formed at the joint; one or more of the cartilage layer, the adhesive layer and the bone layer are porous.

In the present disclosure, the cartilage layer may be prepared by a method conventional in the art, generally by crosslinking curing with a hydrogel composition as a raw material. Wherein, the hydrogel composition and the method of the crosslinking curing are as previously described.

In a preferred embodiment of the present disclosure, the hydrogel composition of the cartilage layer comprises the following components in parts by mass: 1-50 parts of gelatin methacrylate, 0-30 parts of hyaluronic acid methacrylate, 0-30 parts of chondroitin sulfate methacrylate, 0.01-1 part of photoinitiator and gel medium.

Herein, the amount of the gelatin methacrylate is preferably 1-30 parts, more preferably 1-20 parts, more preferably 2-15 parts, more preferably 5-15 parts, for example 8, 10 or 12 parts.

Herein, the amount of the hyaluronic acid methacrylate is preferably 0.1-20 parts, more preferably 0.5-10 parts, more preferably 1-3 parts, for example 1.5 or 2 parts.

Herein, the amount of the chondroitin sulfate methacrylate is preferably 0.1-20 parts, more preferably 0.5-20 parts, more preferably 0.5-5 parts, more preferably 1-3 parts, for example 1 part, 2 parts, 2.5 parts or 3 parts.

Herein, the gelatin methacrylate and the hyaluronic acid methacrylate may have a mass ratio of (1-30):(0.5-10), preferably (2-15):(1-3), for example 5:2.

Herein, the mass ratio of the gelatin methacrylate, the hyaluronic acid methacrylate and the chondroitin sulfate methacrylate may be (1-30):(0.5-10):(0.5-20), preferably (2-15):1:(1-3), for example 10:1:3, 5:2:2, 15:1:1 or 8:1:3.

Herein, the amount of the gel medium may be conventional in the art, preferably such that in the hydrogel composition: 5%-30% of gelatin methacrylate (GelMA), 0.5%-2% of hyaluronic acid methacrylate (HAMA), 0.1%-5% of chondroitin sulfate methacrylate (CSMA), 0.01%-1% of photoinitiator; wherein the percentage is the mass (g) of the components per 100 mL of gel medium.

Herein, the hydrogel composition of the cartilage layer may further comprise a thickener, the thickener being preferably in an amount of 0.1-25 parts. The thickener is as previously described.

When the thickener includes sodium alginate, the amount of the sodium alginate is preferably 0.5-2 parts. When the thickener includes hyaluronic acid, the amount of the hyaluronic acid is preferably 0.5-2 parts. When the thickener includes polyvinylpyrrolidone, the amount of the polyvinylpyrrolidone is preferably 2-10 parts. When the thickener includes gum arabic, the amount of the gum arabic is preferably 0.1-25 parts. When the thickener includes gellan gum, the amount of the gellan gum is preferably 0.1-2 parts. When the thickener includes xanthan gum, the amount of the xanthan gum is preferably 0.1-5 parts.

Wherein, the hydrogel composition of the cartilage layer may further comprise a synthetic photosensitive material. The amount of the synthetic photosensitive material is preferably 5-30 parts. The synthetic photosensitive material is as previously described.

In the present disclosure, preferably, the hydrogel composition of the cartilage layer comprises the following components in parts by mass: 5-15 parts of gelatin methacrylate (GelMA), 0.5-2 parts of hyaluronic acid methacrylate (HAMA), and 0.1-0.5 parts of photoinitiator.

In the present disclosure, preferably, the hydrogel composition of the cartilage layer comprises the following components in parts by mass: 5-15 parts of gelatin methacrylate (GelMA), 0.5-2 parts of hyaluronic acid methacrylate (HAMA), 0.5-3 parts of chondroitin sulfate methacrylate (CSMA), and 0.1-0.5 parts of photoinitiator.

In the present disclosure, preferably, the hydrogel composition of the cartilage layer comprises the following components in parts by mass: 5-15 parts of gelatin methacrylate (GelMA), 1-2 parts of sodium alginate (Alg), and 0.1-0.5 parts of photoinitiator.

In a specific embodiment of the present disclosure, the hydrogel composition of the cartilage layer comprises the following components in parts by mass: 5 parts of gelatin methacrylate (GelMA), 2 parts of hyaluronic acid methacrylate (HAMA), 2 parts of chondroitin sulfate methacrylate (CSMA), and 0.25 parts of photoinitiator.

In a specific embodiment of the present disclosure, the hydrogel composition of the cartilage layer comprises the following components in parts by mass: 10 parts of gelatin methacrylate (GelMA), 1 part of hyaluronic acid methacrylate (HAMA), 3 parts of chondroitin sulfate methacrylate (CSMA), and 0.25 parts of photoinitiator.

In a specific embodiment of the present disclosure, the hydrogel composition of the cartilage layer comprises the following components in parts by mass: 15 parts of gelatin methacrylate (GelMA), 1 part of hyaluronic acid methacrylate (HAMA), 1 part of chondroitin sulfate methacrylate (CSMA), and 0.25 parts of photoinitiator.

In a specific embodiment of the present disclosure, the hydrogel composition of the cartilage layer comprises the following components in parts by mass: 8 parts of gelatin methacrylate (GelMA), 1 part of hyaluronic acid methacrylate (HAMA), 3 parts of chondroitin sulfate methacrylate (CSMA), and 0.25 parts of photoinitiator.

In a specific embodiment of the present disclosure, the hydrogel composition of the cartilage layer comprises the following components in parts by mass: 8 parts of gelatin methacrylate (GelMA), 1 part of hyaluronic acid methacrylate (HAMA), 3 parts of chondroitin sulfate methacrylate (CSMA), 2 parts of sodium alginate, and 0.25 parts of photoinitiator.

In a specific embodiment of the present disclosure, the hydrogel composition of the cartilage layer comprises the following components in parts by mass: 8 parts of gelatin methacrylate (GelMA), 1 part of hyaluronic acid methacrylate (HAMA), 3 parts of chondroitin sulfate methacrylate (CSMA), 10 parts of PEGDA, and 1 part of photoinitiator.

In a specific embodiment of the present disclosure, the hydrogel composition of the cartilage layer comprises the following components in parts by mass: 5 parts of gelatin methacrylate (GelMA), 2 parts of hyaluronic acid methacrylate (HAMA), and 0.5 parts of photoinitiator.

In a specific embodiment of the present disclosure, the hydrogel composition of the cartilage layer comprises the following components in parts by mass: 5 parts of gelatin methacrylate (GelMA), 2 parts of sodium alginate (Alg), and 0.5 parts of photoinitiator.

In the present disclosure, the preparation method for the cartilage layer may be a perfusion method, comprising the following steps: pouring the hydrogel composition of the cartilage layer into a cartilage mold for photocrosslinking to obtain a cured hydrogel; freeze-drying the cured hydrogel to obtain the cartilage layer.

Wherein, the cartilage mold may be designed according to the shape and size of the desired cartilage by conventional methods in the art.

In the present disclosure, the preparation method for the cartilage layer may be a direct 3D printing method, comprising the following steps: 3D printing the hydrogel composition of the cartilage layer and performing photocrosslinking at the same time to obtain a cured hydrogel; freeze-drying the cured hydrogel to obtain the cartilage layer.

In a preferred embodiment, the preparation method for the cartilage layer comprises: printing the hydrogel composition using an extrusion 3D printer with blue light curing, wherein the holding temperature is 30-37° C., the printing ambient temperature is 22-25° C., the printing pressure is 20-40 PSI, the printing speed is 4-8 mm/s, the filling rate is 40%-60%, and the light intensity is 5-20 mW/cm².

In the present disclosure, the preparation method for the cartilage layer is preferably a preparation method for the bionic tissue scaffold as previously described, i.e., a 3D printing demolding method, specifically comprising the following steps of:

S1, carrying out 3D printing with a sacrificial material to obtain a cartilage layer mold; wherein the sacrificial material is a hard high polymer material which can be dissolved in a solvent;

S2, pouring the hydrogel composition of the cartilage layer into the cartilage layer mold for crosslinking to obtain a cured hydrogel-cartilage layer mold complex;

S3, demolding: dissolving the cartilage layer mold with a solvent to obtain a cured hydrogel;

S4, freeze-drying the obtained cured hydrogel to obtain the cartilage layer.

In the above embodiment, the sacrificial material, the hard high polymer material, the 3D printing method, the operation and conditions of the crosslinking, the solvent, and the freeze-drying are all as previously described.

In the present disclosure, the preparation method for the cartilage layer preferably further comprises the step of loading a cartilage promoting component. The method for loading the cartilage promoting component may be conventional in the art. The cartilage promoting component is as previously described. When the cartilage promoting component is a bioactive factor, the method for loading the bioactive factor is generally to soak the cartilage layer in a bioactive factor solution, wherein the concentration of the bioactive factor solution may be 1-200 μg/mL, preferably 5-100 μg/mL.

In the present disclosure, the preparation method for the bone layer may be conventional in the art, preferably 3D printing with the material of the bone layer. Wherein, the material of the bone layer is as previously described. The parameters of the 3D printing can be selected according to the structure and material of the bone layer using conventional methods in the art. The 3D printing is preferably carried out using a fused deposition 3D printer.

In the present disclosure, preferably, the preparation method for the bone layer further comprises the step of loading a bone promoting component. The method of loading the bone promoting component may be conventional in the art, for example: grinding the material of the bone layer into powder and then mixing it with the bone promoting component; or, dissolving the material of the bone layer in a solvent and then mixing it with the bone promoting component, and then allowing the solvent to evaporate. The bone promoting component is as previously described. The solvent may be an organic solvent. When the bone promoting component is a bioactive factor, the method for loading the bioactive factor is generally to soak the bone layer in a bioactive factor solution, wherein the concentration of the bioactive factor solution may be 1-200 μg/mL, preferably 5-100 μg/mL.

In a preferred embodiment, the bone layer is provided by 3D printing with hydroxyapatite-loaded PLA using a fused deposition 3D printer, wherein the print head temperature is 210° C., the platform temperature is 50-60° C., the printing speed is 50-60 mm/s, and the filling rate is 40%-60%.

In a preferred embodiment, the bone layer is provided by 3D printing with PCL loaded with tricalcium phosphate using a fused deposition 3D printer, wherein the printing temperature is 140-150° C., the printing speed is 50-60 mm/s, and the filling rate is 40%-60%.

In a preferred embodiment, the bone layer is provided by 3D printing with GelMA loaded with tricalcium phosphate using a extrusion-type photocuring 3D printer, wherein the holding temperature is 30-35° C., the printing ambient temperature is 22-25° C., the printing pressure is 20-40 PSI, the printing speed is 4-8 mm/s, the filling rate is 40%-60%, and the light intensity is 5-50 mW/cm².

In the present disclosure, the bone layer may also be prepared by crosslinking curing with a hydrogel composition as a raw material. When the bone layer is prepared by crosslinking curing with a hydrogel composition as a raw material, the hydrogel composition and the method of the crosslinking curing are as previously described.

In a preferred embodiment, the hydrogel composition for the bone layer comprises the following components: 5%-30% of gelatin methacrylate (GelMA), 2.5%-50% of bioactive glass and 0.01%-1% of photoinitiator; wherein the percentage is the mass (g) of the components per 100 mL of gel medium.

The connection of the present disclosure is generally a connection between the bone layer and the cartilage by means of a medical glue. The operating conditions of the connection can be determined according to the method of use of the medical glue used. The medical glue is as previously described.

In certain embodiments, the osteochondral scaffold is prepared using the preparation method for the bionic tissue scaffold as previously described, i.e., a 3D printing demolding method for osteochondral one-piece molding, specifically comprising the following steps of:

S1, carrying out 3D printing with a sacrificial material to obtain an osteochondral scaffold mold; wherein the sacrificial material is a hard high polymer material which can be dissolved in a solvent;

S2, sequentially pouring the hydrogel composition of the bone layer, the adhesive layer and the cartilage layer into the osteochondral scaffold mold for crosslinking to obtain a cured hydrogel-osteochondral scaffold mold complex;

S3, demolding: dissolving the osteochondral scaffold mold with a solvent to obtain a cured hydrogel;

S4, freeze-drying the obtained cured hydrogel to obtain the osteochondral scaffold.

In the above embodiment, preferably, the hydrogel composition of the bone layer comprises the following components: 5%-30% of gelatin methacrylate (GelMA), 2.5%-50% of bioactive glass and 0.01%-1% of photoinitiator; the hydrogel composition of the adhesive layer comprises the following components: 5%-30% of gelatin methacrylate (GelMA), 10%-20% of bioactive glass and 0.01%-1% of photoinitiator; the hydrogel composition of the cartilage layer comprises the following components: 5%-30% of gelatin methacrylate (GelMA), 0.5%-2% of hyaluronic acid methacrylate (HAMA), 0.5%-5% of chondroitin sulfate methacrylate (CSMA), and 0.01%-1% of photoinitiator; wherein the percentage is the mass (g) of the components per 100 mL of gel medium.

In the above embodiment, the sacrificial material, the hard high polymer material, the 3D printing method, the operation and conditions of the crosslinking, the solvent, and the freeze-drying are all as previously described.

The present disclosure also provides an application of the osteochondral scaffold in the repair of cartilage defects.

In the present disclosure, the cartilage defect may be an osteochondral composite defect or a simple cartilage defect. The osteochondral defect may be located in the knee joint, hip joint or shoulder joint.

In repairing an osteochondral composite defect, the osteochondral scaffold may be used by drilling down to the bone layer at the osteochondral composite defect, reaching the bone marrow cavity, removing the excess bone layer matrix, and placing the osteochondral scaffold material into the osteochondral composite defect. At this point, the bone marrow flowing out of the bone layer is rich in mesenchymal stem cells, which are captured in the scaffold as they flow through the osteochondral scaffold material. After the operation is completed, the wound is sutured and the repair of the osteochondral composite defect is completed.

In repairing a simple cartilage defect, the osteochondral scaffold may be used by placing the cartilage layer of the osteochondral scaffold in the defect and performing microfracture treatment on the cartilage defect to the point where the bone marrow flows out. At this point, the bone marrow emerging from the bone layer is rich in mesenchymal stem cells, which are captured in the scaffold as they flow through the osteochondral scaffold. After the operation is completed, the wound is sutured and the repair of the simple cartilage defect is completed.

The osteochondral scaffold of the present disclosure has the following advantages: (1) The osteochondral scaffold of the present disclosure has a refined through-hole structure to ensure vertical and horizontal penetration, which facilitates the scaffold to capture cells adequately when filling the osteochondral defects, and also facilitates the transport of nutrients and metabolic wastes, thus facilitating the defect repair. Further, the differentiation and growth of stem cells into chondrocytes and osteocytes can be induced by selecting suitable cartilage layer materials and bone layer materials. (2) The osteochondral scaffold of the present disclosure has an excellent adhesive layer structure, which can firmly connect the cartilage layer and the bone layer to achieve the purpose of integration; the adhesive layer acts as a transition layer of the bone layer to prevent the ossification of the cartilage layer. The osteochondral scaffold of the present disclosure can realize the full-layer repair of osteochondral defects. (3) The osteochondral scaffold of the present disclosure has a simple clinical operation mode and practicability, which provides a new and effective solution for the repair of cartilage defects and osteochondral composite defects in clinical practice.

The present disclosure also provides a hydrogel composition for a bionic cartilage scaffold comprising the following components in parts by mass: 1-50 parts of gelatin methacrylate (GelMA), 0.1-30 parts of hyaluronic acid methacrylate (HAMA), 0.1-30 parts of chondroitin sulfate methacrylate (CSMA), 0.01-1 part of photoinitiator and gel medium.

In the present disclosure, the amount of the gelatin methacrylate is preferably 1-20 parts, more preferably 5-15 parts, for example 8, 10 or 12 parts.

In the present disclosure, the methacrylation degree of the gelatin methacrylate may be 30%-100%, preferably 40%-80%.

In the present disclosure, the amount of the hyaluronic acid methacrylate is preferably 0.1-10 parts, more preferably 0.5-10 parts, further more preferably 0.5-2 parts, for example 0.5 parts, 1 part, 1.5 parts.

In the present disclosure, the molecular weight of the hyaluronic acid methacrylate may be 1-8000 kDa, preferably 100-1000 kDa, more preferably 500-950 kDa.

In the present disclosure, the methacrylation degree of the hyaluronic acid methacrylate may be 20%-60%, preferably 30%-50%.

In the present disclosure, the amount of the chondroitin sulfate methacrylate is preferably 0.1-10 parts, more preferably 0.5-10 parts, further more preferably 0.5-3 parts, for example 1 part, 2 parts or 2.5 parts.

In the present disclosure, the molecular weight of the chondroitin sulfate methacrylate may be 5-50 kDa, preferably 10-40 kDa.

In the present disclosure, the methacrylation degree of the chondroitin sulfate methacrylate may be 20%-60%, preferably 30%-50%.

In the present disclosure, the mass ratio of the gelatin methacrylate, the hyaluronic acid methacrylate and the chondroitin sulfate methacrylate may be (2-15):(0.5-5):(1-5), preferably (2-15):1:(1-3), for example 10:1:3, 5:2:2, 15:1:1 or 8:1:3.

In the present disclosure, the type of the photoinitiator is as previously described. The amount of the photoinitiator is preferably 0.1-0.5 parts, for example 0.25 parts.

In the present disclosure, preferably, the hydrogel composition for a bionic cartilage scaffold comprises the following components in parts by mass: 5-15 parts of gelatin methacrylate (GelMA), 0.5-10 parts of hyaluronic acid methacrylate (HAMA), 0.5-10 parts of chondroitin sulfate methacrylate (CSMA), and 0.1-0.5 parts of photoinitiator.

In a preferred embodiment of the present disclosure, the hydrogel composition for a bionic cartilage scaffold comprises the following components in parts by mass: 5 parts of gelatin methacrylate (GelMA), 2 parts of hyaluronic acid methacrylate (HAMA), 2 parts of chondroitin sulfate methacrylate (CSMA), and 0.25 parts of photoinitiator.

In a preferred embodiment of the present disclosure, the hydrogel composition for a bionic cartilage scaffold comprises the following components in parts by mass: 10 parts of gelatin methacrylate (GelMA), 1 part of hyaluronic acid methacrylate (HAMA), 3 parts of chondroitin sulfate methacrylate (CSMA), and 0.25 parts of photoinitiator.

In a preferred embodiment of the present disclosure, the hydrogel composition for a bionic cartilage scaffold comprises the following components in parts by mass: 15 parts of gelatin methacrylate (GelMA), 1 part of hyaluronic acid methacrylate (HAMA), 1 part of chondroitin sulfate methacrylate (CSMA), and 0.25 parts of photoinitiator.

In a preferred embodiment of the present disclosure, the hydrogel composition for a bionic cartilage scaffold comprises the following components in parts by mass: 8 parts of gelatin methacrylate (GelMA), 1 part of hyaluronic acid methacrylate (HAMA), 3 parts of chondroitin sulfate methacrylate (CSMA), and 0.25 parts of photoinitiator.

In a preferred embodiment of the present disclosure, the hydrogel composition for a bionic cartilage scaffold comprises the following components in parts by mass: 8 parts of gelatin methacrylate (GelMA), 1 part of hyaluronic acid methacrylate (HAMA), 3 parts of chondroitin sulfate methacrylate (CSMA), 2 parts of sodium alginate, and 0.25 parts of photoinitiator.

In a preferred embodiment of the present disclosure, the hydrogel composition for a bionic cartilage scaffold comprises the following components in parts by mass: 8 parts of gelatin methacrylate (GelMA), 1 part of hyaluronic acid methacrylate (HAMA), 3 parts of chondroitin sulfate methacrylate (CSMA), 10 parts of PEGDA, and 1 part of photoinitiator.

In the present disclosure, the hydrogel composition for a bionic cartilage scaffold may further comprise a thickener, the thickener being preferably in an amount of 0.1-25 parts. The type of the thickener is as previously described.

Herein, when the thickener includes sodium alginate, the amount of the sodium alginate is preferably 1-2 parts. When the thickener includes hyaluronic acid, the amount of the hyaluronic acid is preferably 0.5-2 parts. When the thickener includes polyvinylpyrrolidone, the amount of the polyvinylpyrrolidone is preferably 2-10 parts. When the thickener includes gum arabic, the amount of the gum arabic is preferably 0.1-25 parts. When the thickener includes gellan gum, the amount of the gellan gum is preferably 0.1-2 parts. When the thickener includes xanthan gum, the amount of the xanthan gum is preferably 0.1-1 parts.

In the present disclosure, the hydrogel composition for a bionic cartilage scaffold may further comprise a synthetic photosensitive material. The amount of the synthetic photosensitive material is preferably 5-30 parts. The type of the synthetic photosensitive material is as previously described.

In the present disclosure, the gel medium is as previously described. The amount of the gel medium may be conventional in the art, preferably such that in the hydrogel composition for a bionic cartilage scaffold: 5%-20% of gelatin methacrylate (GelMA), 0.1%-3% of hyaluronic acid methacrylate (HAMA), 0.1%-5% of chondroitin sulfate methacrylate (CSMA), 0.01%-1% of photoinitiator; wherein the percentage is the mass (g) of the components per 100 mL of gel medium.

The present disclosure also provides a preparation method for a bionic cartilage scaffold, which is obtained by photocrosslinking curing using the hydrogel composition for the bionic cartilage scaffold as a raw material.

In the present disclosure, the preparation method for the bionic cartilage scaffold is preferably a preparation method for the bionic tissue scaffold as previously described, i.e., a 3D printing demolding method, specifically comprising the following steps of:

S1, carrying out 3D printing with a sacrificial material to obtain an bionic cartilage scaffold mold; wherein the sacrificial material is a hard high polymer material which can be dissolved in a solvent;

S2, pouring the hydrogel composition of the cartilage layer into the bionic cartilage scaffold mold for crosslinking to obtain a cured hydrogel-bionic cartilage scaffold mold complex;

S3, demolding: dissolving the bionic cartilage scaffold mold with a solvent to obtain a cured hydrogel;

S4, freeze-drying the obtained cured hydrogel to obtain the bionic cartilage scaffold mold.

In the above embodiment, the sacrificial material, the hard high polymer material, the 3D printing method, the operation and conditions of the crosslinking, the solvent, and the freeze-drying are all as previously described.

In the present disclosure, the preparation method for the bionic cartilage scaffold may be a perfusion method, the perfusion method being operated as previously described.

In the present disclosure, the preparation method for the bionic cartilage scaffold may be a direct 3D printing method, the direct 3D printing method being operated as previously described.

In the above preparation method for the bionic cartilage scaffold, the photocrosslinking curing is as previously described.

The present disclosure also provides a bionic cartilage scaffold, which is prepared by the preparation method for the bionic cartilage scaffold.

The present disclosure also provides an application of the hydrogel composition for a bionic cartilage scaffold or the bionic cartilage scaffold in osteochondral tissue engineering.

The hydrogel composition for the bionic cartilage scaffold provided by the present disclosure can be used to prepare the bionic cartilage scaffold by methods such as perfusion or 3D printing, the preparation method being simple and feasible. The prepared bionic cartilage scaffold has the following advantages: (1) good biocompatibility; (2) degradable and replaced by regenerated cartilage; (3) inhibition of osteogenesis; (4) induction of cartilage formation; (5) high-precision through-hole bionic cartilage scaffold can be obtained by 3D printing demolding method.

On the basis of common knowledge in the art, each of the above preferred conditions can be combined in any way to obtain each preferred embodiment of the present disclosure.

### BRIEF DESCRIPTION OF THE DRAWINGS

FIG. 1 is a mold design drawing of the cartilage scaffold in Embodiments 1-3 of the present disclosure.

FIG. 2 is a microscopic photograph of the mold of the cartilage scaffold in Embodiment 1 of the present disclosure.

FIG. 3 is a microscopic photograph of the cartilage scaffold in Embodiment 1 of the present disclosure.

FIG. 4 is a camera photograph of the mold of the cartilage scaffold in Embodiment 2 of the present disclosure.

FIG. 5 is a camera photograph of the cartilage scaffold in Embodiment 2 of the present disclosure.

FIG. 6 is a mold design drawing of the bone scaffold in Embodiment 4 of the present disclosure.

FIG. 7 is a camera photograph of the mold of the bone scaffold in Embodiment 4 of the present disclosure.

FIG. 8 is a camera photograph of the cured hydrogel in Embodiment 4 of the present disclosure after rehydration.

FIG. 9 is a mold design drawing of the nerve conduit scaffold in Embodiment 5 of the present disclosure.

FIG. 10 is a physical picture of the mold printing of the nerve conduit scaffold in Embodiment 5 of the present disclosure.

FIG. 11 is a diagram of the cured hydrogel-mold complex of the nerve conduit scaffold in Embodiment 5 of the present disclosure.

FIG. 12 is a picture of the conduit scaffold in Embodiment 5 of the present disclosure after demolding.

FIG. 13 is a mold design drawing of the skin scaffold in Embodiment 6 of the present disclosure.

FIG. 14 is a physical picture of the mold printing of the skin scaffold in Embodiment 6 of the present disclosure.

FIG. 15 is a diagram of the cured hydrogel-mold complex of the skin scaffold in Embodiment 6 of the present disclosure.

FIG. 16 is a picture of the skin scaffold in Embodiment 6 of the present disclosure after demolding.

FIG. 17 is a mold design drawing of the muscle scaffold in Embodiment 7 of the present disclosure.

FIG. 18 is a physical picture of the mold printing of the muscle scaffold in Embodiment 7 of the present disclosure.

FIG. 19 is a diagram of the cured hydrogel-mold complex of the muscle scaffold in Embodiment 7 of the present disclosure.

FIG. 20 is a picture of the muscle scaffold in Embodiment 7 of the present disclosure after demolding.

FIG. 21 is a microscopic photograph of MSC cells cultured on the cartilage scaffold in Embodiment 1 of the present disclosure.

FIG. 22 is a structure schematic diagram of the osteochondral scaffold in Embodiments 8-11 of the present disclosure.

FIG. 23 is a camera photograph of the osteochondral scaffold in Embodiment 8 of the present disclosure.

FIG. 24 is a microscopic photograph of the osteochondral scaffold in Embodiment 8 of the present disclosure.

FIG. 25 is a microscopic photograph of MSC cells cultured on the osteochondral scaffold in Effect Embodiment 2 of the present disclosure.

FIG. 26 is a general observation photograph of the injured joints in group (a) in Effect Embodiment 3 of the present disclosure.

FIG. 27 is a general observation photograph of the injured joints in group (b) in Effect Embodiment 3 of the present disclosure.

FIG. 28 is a general observation photograph of the injured joints in group (c) in Effect Embodiment 3 of the present disclosure.

FIG. 29 shows the variation of modulus of the hydrogel with time under light in Effect Embodiment 4 of the present disclosure.

FIG. 30 shows the stress-strain curve of the cured hydrogel scaffold in Effect Embodiment 5 of the present disclosure.

FIG. 31 shows the survival of cells after 7 days of culture in the cured hydrogel in Effect Embodiment 6 of the present disclosure (live cells green-stained, dead cells red-stained).

FIG. 32 is a general observation photograph in Effect Embodiment 7 of the present disclosure.

FIG. 33 is a diagram of a tissue section in Effect Embodiment 7 of the present disclosure.

### DETAILED DESCRIPTION OF THE PREFERRED EMBODIMENT

The present disclosure is further illustrated below by means of embodiments, but the present disclosure is not thereby limited to the scope of the embodiments. Experimental methods for which specific conditions are not specified in the following embodiments are selected according to conventional methods and conditions, or according to the trade description.

### Embodiment 1

A preparation method for a cartilage scaffold, comprising the following steps:

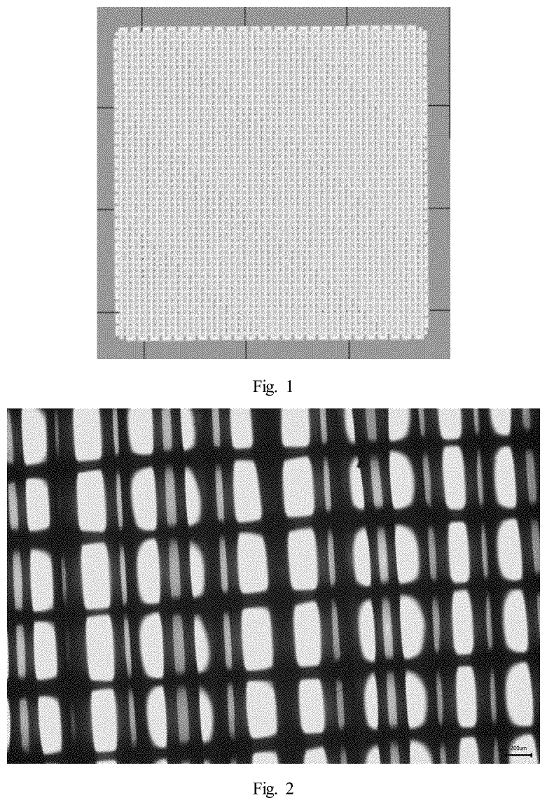

S1, A mold was designed according to the desired cartilage scaffold (as shown in FIG. 1), the mold was printed by fused deposition using PLA as the sacrificial material (as shown in FIG. 2);

S2, the hydrogel composition was poured into the mold, realizing photocrosslinking curing under blue light irradiation with a wavelength of 405 nm and an intensity of 10 mW/cm², and then immersed in 0.1 M CaCl₂ solution for chemical crosslinking curing to obtain a cured hydrogel-mold complex;

Herein, the hydrogel composition was prepared as follows: 0.2 g of sodium alginate (Alg), 0.5 g of GelMA and 50 mg of LAP were weighed and dissolved in 10 mL of PBS solution (pH=7.4), and prepared at 37° C.;

S3, the cured hydrogel-mold complex was immersed in dichloromethane, which dissolved the mold;

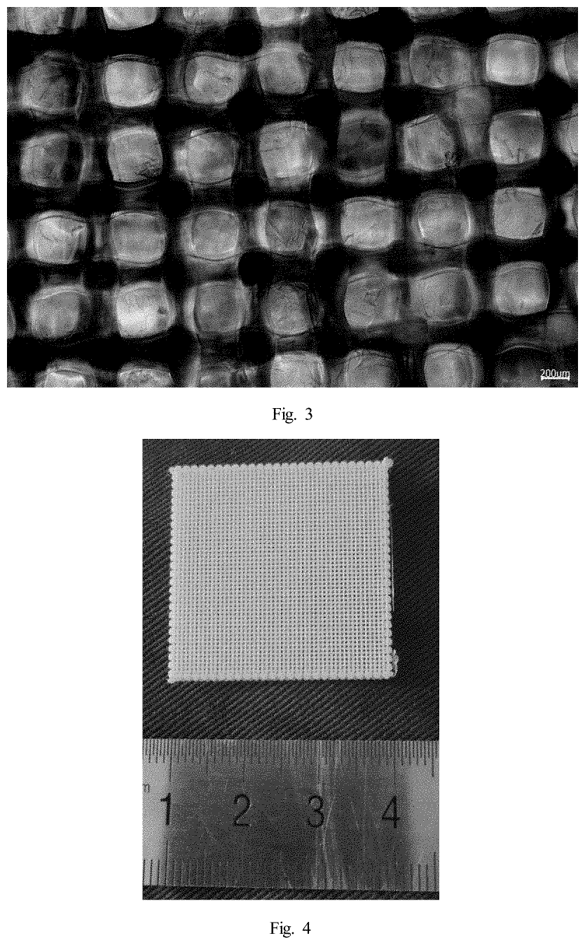

S4, the obtained cured hydrogel was pre-cooled in a −20° C. refrigerator for 2 hours, and then freeze-dried in a freeze-dryer for 8 hours to obtain a cartilage scaffold (as shown in FIG. 3).

### Embodiment 2

A preparation method for a cartilage scaffold, comprising the following steps:

S1, A mold was designed according to the desired cartilage scaffold (as shown in FIG. 1), the mold was printed by fused deposition using PETG as the sacrificial material (as shown in FIG. 4);

S2, the hydrogel composition was poured into the mold, realizing photocrosslinking curing under blue light irradiation with a wavelength of 405 nm and an intensity of 10 mW/cm², a curing hydrogel-mold complex was obtained;

herein, the hydrogel composition was prepared as follows: 0.5 g of GelMA, 0.2 g of HAMA and 50 mg of LAP were weighed and dissolved in 10 mL of PBS solution (pH=7.4), and prepared at 37° C.;

S3, the cured hydrogel-mold complex was immersed in dichloromethane, which dissolved the mold;

S4, the obtained cured hydrogel was pre-cooled in a −20° C. refrigerator for 2 hours, and then freeze-dried in a freeze-dryer for 20 hours to obtain a cartilage scaffold (as shown in FIG. 5).

### Embodiment 3

A preparation method for a cartilage scaffold, comprising the following steps:

S1, A mold was designed according to the desired cartilage scaffold (as shown in FIG. 1), the mold was printed by the photocuring method (LCD) using PLA bio-based photosensitive resin (eSUN New Material Co., Ltd.) as the sacrificial material.

S2, the hydrogel composition was poured into the mold, realizing photocrosslinking curing under blue light irradiation with a wavelength of 405 nm and an intensity of 10 mW/cm², a cured hydrogel-mold complex was obtained;

wherein, the hydrogel composition was prepared as follows:

1 g of GelMA, 0.2 g of HAMA, 0.2 g of CSMA and 50 mg of LAP were weighed and dissolved in 10 mL of PBS solution (pH=7.4), and prepared at 37° C.;

S3, the cured hydrogel-mold complex was immersed in dichloromethane, which dissolved the mold;

S4, the obtained cured hydrogel was pre-cooled in a −20° C. refrigerator for 2 hours, and then freeze-dried in a freeze-dryer for 12 hours to obtain a cartilage scaffold.

### Embodiment 4

A preparation method for a bone scaffold, comprising the following steps:

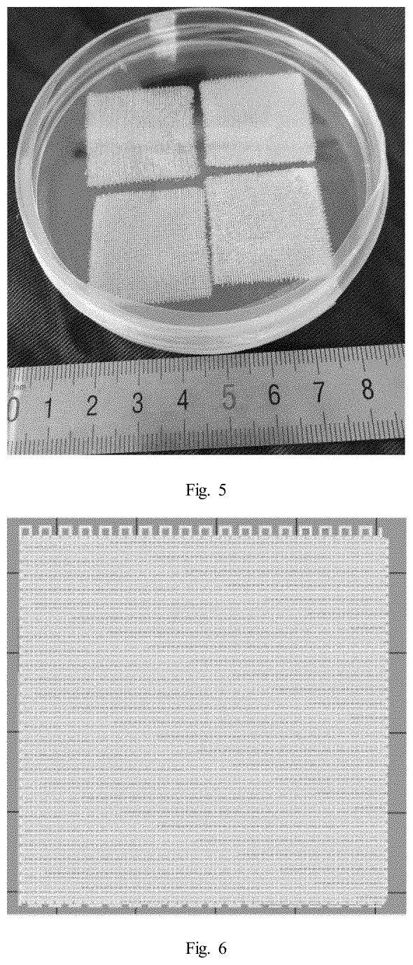

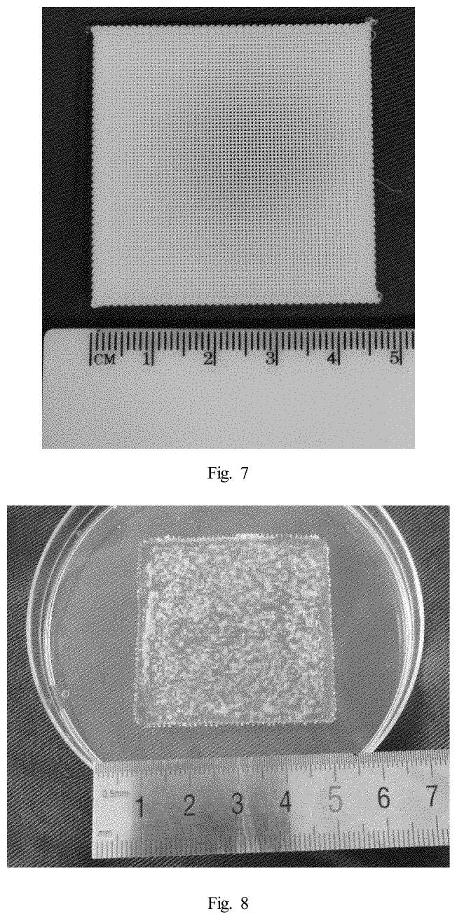

S1, A mold was designed according to the desired cartilage scaffold (as shown in FIG. 6), the mold was printed by fused deposition using PVA as the sacrificial material (as shown in FIG. 7);

S2, the hydrogel composition was poured into the mold, realizing photocrosslinking curing under blue light irradiation with a wavelength of 405 nm and an intensity of 10 mW/cm², and then immersed in 0.1 M CaCl₂ solution for physical crosslinking curing to obtain a cured hydrogel-mold complex;

herein, the hydrogel composition was prepared as follows: 0.2 g of sodium alginate (Alg), 0.5 g of GelMA, 0.1 g of hydroxyapatite (HAp) and 30 mg of LAP were weighed and dissolved in 10 mL of PBS solution (pH=7.4), and prepared at 37° C.;

S3, the cured hydrogel-mold complex was immersed in purified water to dissolve the mold;

S4, the obtained cured hydrogel was pre-cooled in a −20° C. refrigerator for 2 hours, and then freeze-dried in a freeze-dryer for 20 hours to obtain a bone scaffold. The photo after rehydration is shown in FIG. 8.

### Embodiment 5

A preparation method for a nerve conduit scaffold, comprising the following steps:

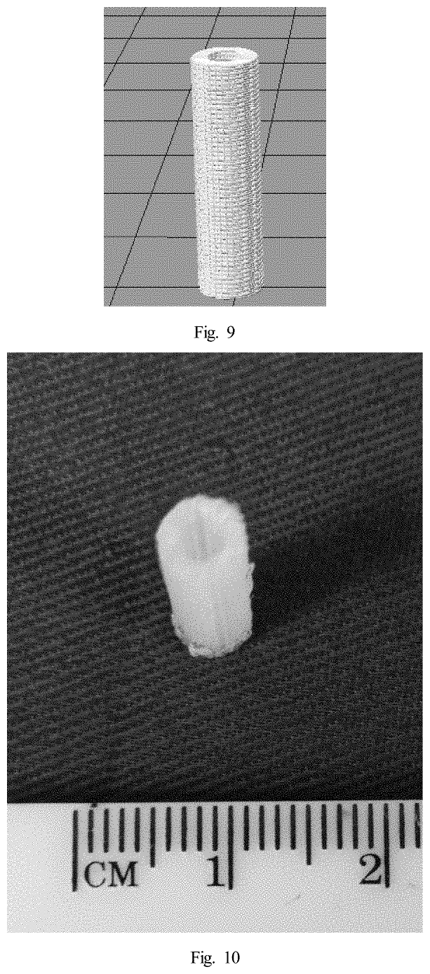

S1, A mold was designed according to the desired nerve conduit scaffold (as shown in FIG. 9), the mold was printed by fused deposition using PLA as the sacrificial material (as shown in FIG. 10);

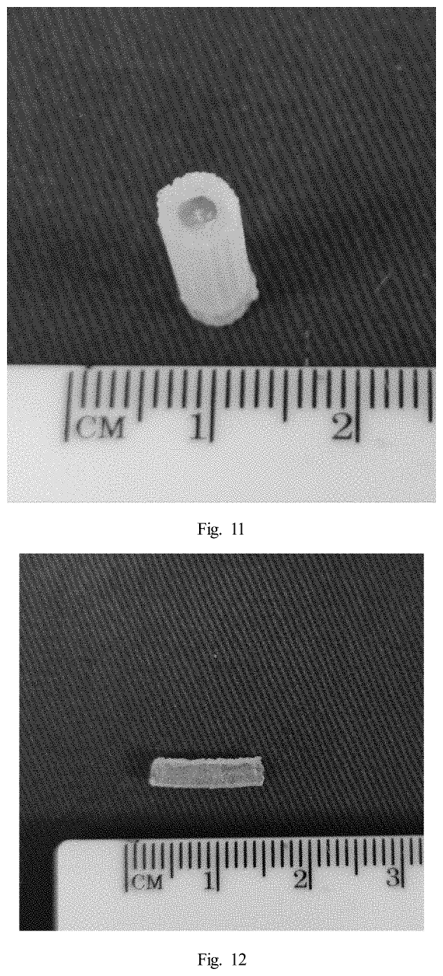

S2, the hydrogel composition was poured into the mold, realizing photocrosslinking curing under blue light irradiation with a wavelength of 405 nm and an intensity of 10 mW/cm², and then immersed in 0.1 M CaCl₂ solution for physical crosslinking curing to obtain a cured hydrogel-mold complex (as shown in FIG. 11);

herein, the hydrogel composition was prepared as follows: 0.2 g of sodium alginate (Alg), 0.5 g of GelMA and 25 mg of LAP were weighed and dissolved in 10 mL of PBS solution (pH=7.4), and prepared at 37° C.;

S3, the cured hydrogel-mold complex was immersed in dichloromethane, which dissolved the mold (as shown in FIG. 12);

S4, the obtained cured hydrogel was pre-cooled in a −20° C. refrigerator for 2 hours, and then freeze-dried in a freeze-dryer for 20 hours to obtain a nerve conduit scaffold.

### Embodiment 6

A preparation method for a skin scaffold, comprising the following steps:

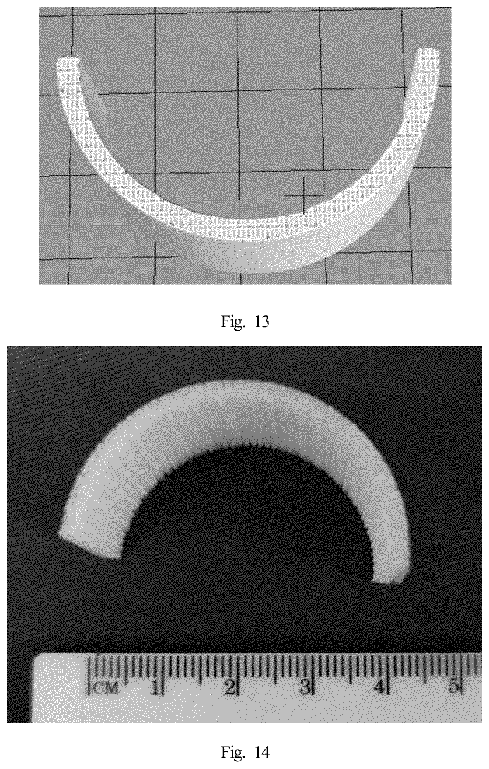

S1, A mold was designed according to the desired skin scaffold (as shown in FIG. 13), the mold was printed by fused deposition using PCL as the sacrificial material (as shown in FIG. 14);

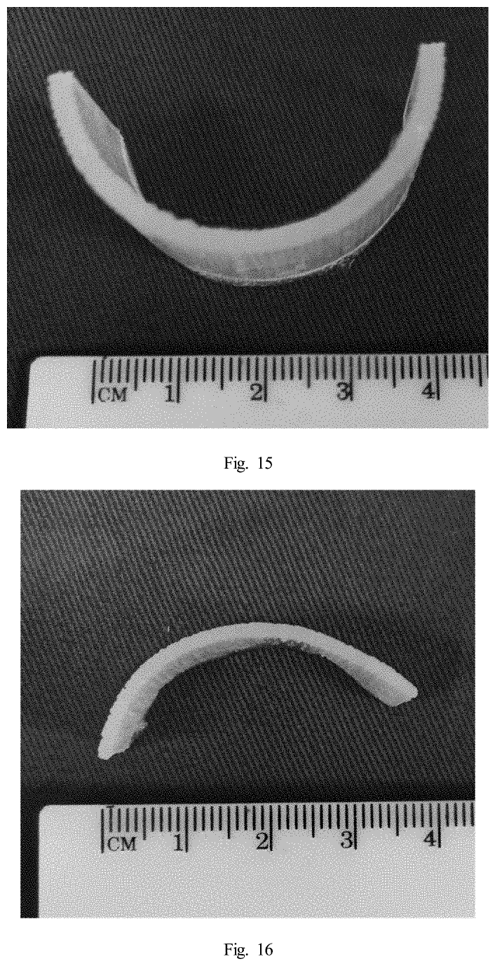

S2, the hydrogel composition was poured into the mold, realizing photocrosslinking curing under blue light irradiation with a wavelength of 405 nm and an intensity of 10 mW/cm², a cured hydrogel-mold complex was obtained (as shown in FIG. 15);

herein, the hydrogel composition was prepared as follows: 0.2 g of Col I MA, 1.0 g of GelMA, and 10 mg of LAP were weighed and dissolved in 10 mL of PBS solution (pH=7.4), and prepared at 37° C.;

S3, the cured hydrogel-mold complex was immersed in dichloromethane, which dissolved the mold (as shown in FIG. 16);

S4, the obtained cured hydrogel was pre-cooled in a −20° C. refrigerator for 2 hours, and then freeze-dried in a freeze-dryer for 20 hours to obtain a skin scaffold.

### Embodiment 7

A preparation method for a muscle scaffold, comprising the following steps:

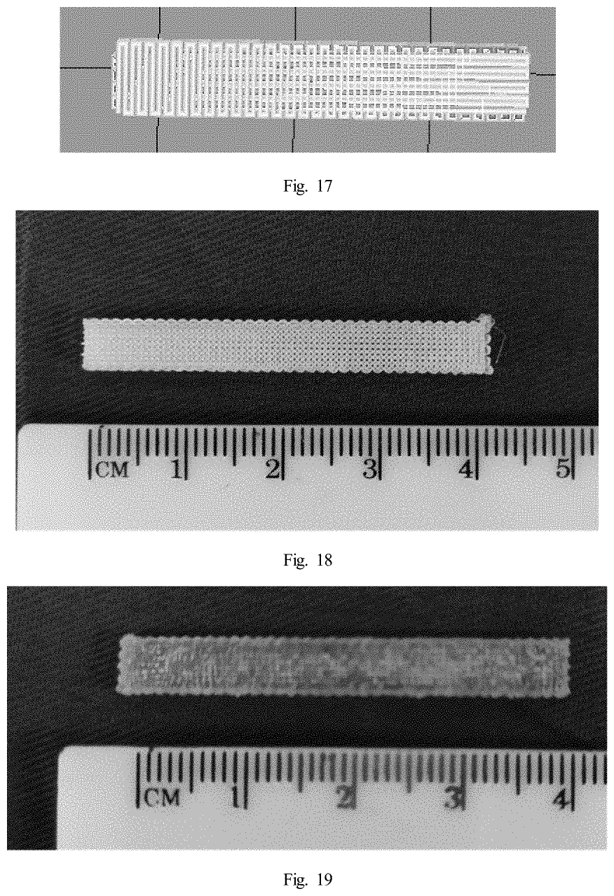

S1, A mold was designed according to the desired muscle scaffold (as shown in FIG. 17), the mold was printed by fused deposition using PLA as the sacrificial material (as shown in FIG. 18);

S2, the hydrogel composition was poured into the mold, realizing photocrosslinking curing under blue light irradiation with a wavelength of 405 nm and an intensity of 10 mW/cm², a cured hydrogel-mold complex was obtained (as shown in FIG. 19);

herein, the hydrogel composition was prepared as follows: 0.2 g of HAMA, 1.0 g of GelMA, and 10 mg of LAP were weighed and dissolved in 10 mL of PBS solution (pH=7.4), and prepared at 37° C.;

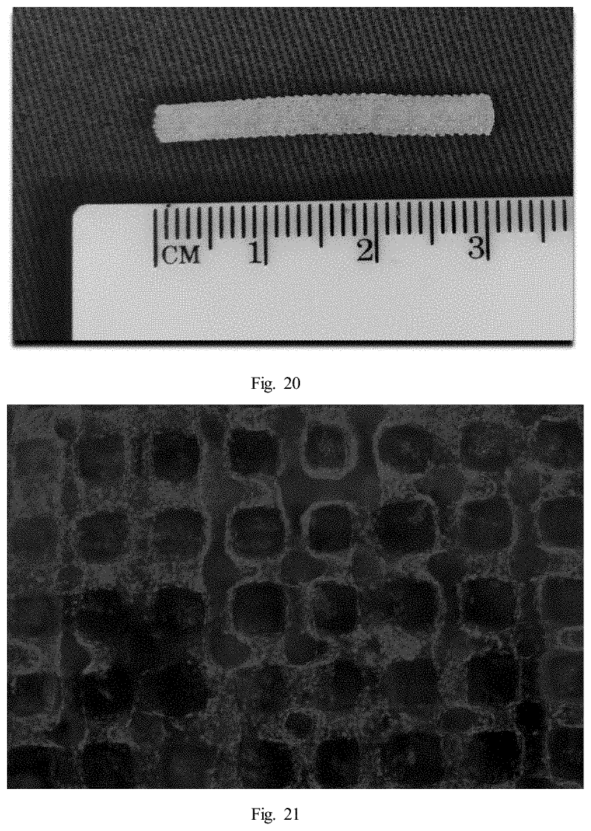

S3, the cured hydrogel-mold complex was immersed in dichloromethane, which dissolved the mold (as shown in FIG. 20);

S4, the obtained cured hydrogel was pre-cooled in a −20° C. refrigerator for 2 hours, and then freeze-dried in a freeze-dryer for 20 hours to obtain a muscle scaffold.

### Effect Embodiment 1: Cytocompatibility Test

Taking the cartilage scaffold prepared in Embodiment 1 as an example, the mesenchymal stem cells (MSCs) were planted on the scaffold material, the culture medium was added, and cultured at a condition of 37° C./5% CO₂ for 24 hours. Before the test, the cell culture medium was aspirated and washed several times with PBS, 1 mL of live/dead cell double staining reagent (10 μM of calcein and 15 μM of ethidium dimer dissolved in 5 mL of PBS) was added, incubated with the cells at 37° C. for 30 min, and then the adhesion and survival of the cells inside the scaffold material were observed using confocal fluorescence microscope. Live cells exhibited calcein staining activity and emitted green fluorescence at 433 nm; dead cells were stained by ethidium bromide and emitted red fluorescence under 543 nm excitation. As seen in FIG. 21, this type of scaffold material has good cytocompatibility and is able to grow into the through-hole structure of the scaffold material.

### Embodiment 8

### 1. Preparation of Cartilage Layer

(1) Synthesis of gelatin methacrylate (GelMA): gelatin (1 g) was dissolved in 10 mL of PBS (pH=7.4), heated to 50° C. and stirred until completely dissolved, 0.5 mL of methacrylic anhydride was added, reacted for 2-3 hours, after the reaction, the reaction solution was diluted with 40 mL of PBS, then poured into a dialysis bag (mWCO 7000), dialyzed with deionized water for 2-3 days, and freeze-dried to obtain the gelatin methacrylate (0.9 g). According to the hydrogen nuclear magnetic resonance (¹H NMR), the integrated area of the standard peak of phenylalanine (7.1-7.4 ppm) was selected as 1, the percentage decrease in peak area of the lysine signal at 2.8-2.95 ppm before and after gelatin modification was calculated, and the methacrylation degree of gelatin methacrylate was 65%.

(2) Synthesis of hyaluronic acid methacrylate (HAMA): hyaluronic acid (1 g, 900 kDa) was dissolved in 100 mL of deionized water, cooled to 0-4° C., 5 mL of methacrylic anhydride was added, 5 mL of 5M NaOH aqueous solution was slowly added dropwise, reacted for 24 hours, the reaction solution was poured into a dialysis bag (mWCO 7000), dialyzed with deionized water for 2-3 days, freeze-dried to obtain the hyaluronic acid methacrylate (0.9 g). According to the hydrogen nuclear magnetic resonance (¹H NMR), the degree of methacrylation of HAMA was calculated to be 40% (methacrylation degree of HAMA=peak area of methacrylate-vinyl at 5.6 ppm/peak area of N-acetyl glucosamine at 1.9 ppm*100%).

(3) Preparation of 5% GelMA/2% HAMA/0.5% LAP hydrogel composition: 0.05 g of GelMA, 0.02 g of HAMA and 5mg of LAP were weighed and dissolved in 1 mL of PBS solution (pH=7.4), 5% GelMA/2% HAMA/0.5% LAP was prepared at 37° C.

(4) Preparation of GelMA/HAMA cartilage layer by perfusion method:

The hydrogel composition was poured into a prefabricated cylindrical mold (5 mm in diameter and 3 mm in height), photocrosslinking was realized under the irradiation of a light source with a wavelength of 405 nm and an intensity of 10 mW/cm² to obtain a GelMA/HAMA photocrosslinked cured hydrogel; the prepared GelMA/HAMA photocrosslinked cured hydrogel was frozen in a −20° C. refrigerator for 2 hours, and then freeze-dried in a freeze-dryer to obtain a GelMA/HAMA cartilage layer.

(5) Loading of TGFβ: The above GelMA/HAMA cartilage layer was immersed in 10 μg/mL TGFβ solution, after 12 hours of full adsorption, it was frozen in a −20° C. refrigerator for 2 hours, and then freeze-dried in a freeze-dryer to obtain a TGFβ-loaded cartilage layer, which was denoted as TGFβ-GelMA/HAMA cartilage layer.

### 2. Preparation of Cartilage Layer

(1) Preparation of hydroxyapatite-loaded polylactic acid (HAP/PLA) high polymer material

5 g of polylactic acid (PLA) was weighed and dissolved in 20 mL of dichloromethane until it was completely dissolved, 1 g of hydroxyapatite (HAP) was added, stirred until the solution was homogeneous, placed in a fume hood until dichloromethane evaporated, dried in a vacuum drying oven for 24 hours, crushed into powder with a pulverizer, and made into a 1.75 mm wire by a wire drawing machine to obtain a HAP/PLA high polymer material.

(2) 3D printing of HAP/PLA high polymer material

The HAP/PLA high polymer material was printed by a fused deposition 3D printer (printing temperature: 210° C.; platform temperature: 50° C.; printing speed: 60 mm/s; filling rate: 50%, layer height: 0.1 mm) to obtain a HAP/PLA bone layer. The obtained HAP/PLA bone layer was a cylinder (5 mm in diameter and 3 mm in height) with a porosity of 50% and a pore diameter of 250 μm.

### 3. Preparation of Osteochondral Scaffold

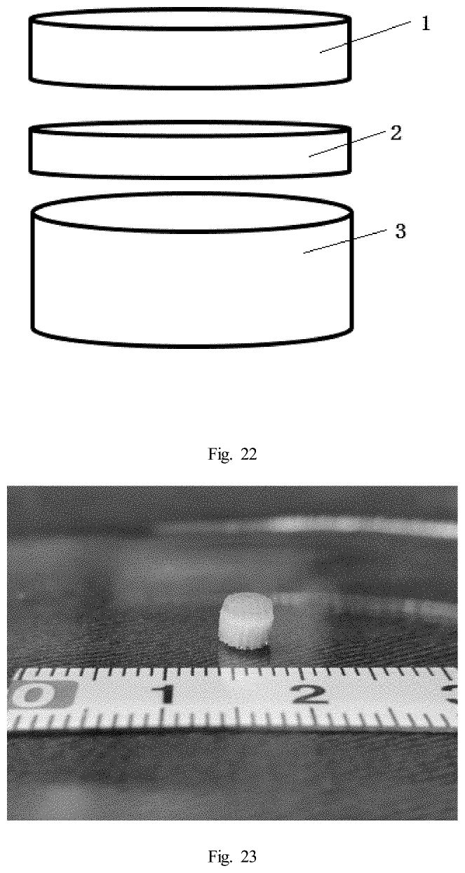

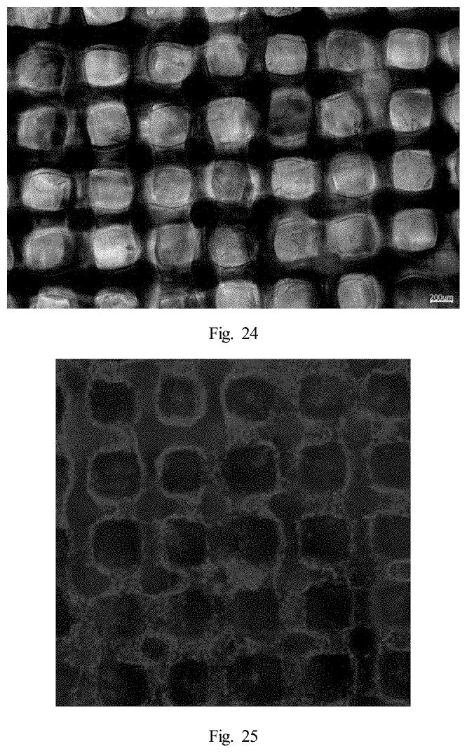

The above TGFβ-GelMA/HAMA cartilage layer and HAP/PLA bone layer were connected with medical glue Golden Elephant, an adhesive layer was formed at the joint, the thickness of the adhesive layer was about 100 μm, and a TGFβ-GelMA/HAMA-HAP/PLA osteochondral scaffold was obtained, its structure schematic diagram is shown in FIG. 22, with cartilage layer 1, adhesive layer 2 and bone layer 3 from top to bottom; the physical camera photo is shown in FIG. 23; the microstructure was observed through a microscope as shown in FIG. 24.

In this embodiment, the GelMA/HAMA-HAP/PLA osteochondral scaffold is obtained if TGFβ is not loaded.

### Embodiment 9

### 1. Preparation of Cartilage Layer

(1) Synthesis of gelatin methacrylate (GelMA): same as Embodiment 1.

(2) Synthesis of hyaluronic acid methacrylate (HAMA): same as Embodiment 1.

(3) Preparation of 5% GelMA/2% HAMA/0.5% LAP hydrogel composition: same as Embodiment 1.

(4) Preparation of cartilage layer by direct 3D printing method:

The above hydrogel composition was printed using an extrusion 3D printer with blue light curing (holding temperature: 37° C.; platform temperature: 22° C.; printing pressure: 20 PSI; printing speed: 5 mm/s; filling rate: 50%); the prepared GelMA/HAMA photocrosslinked cured hydrogel was frozen in a −20° C. refrigerator for 2 hours, and then freeze-dried in a freeze-dryer to obtain a GelMA/HAMA cartilage layer. The obtained GelMA/HAMA cartilage layer was a cylinder (5 mm in diameter and 1 mm in height) with a porosity of 50% and a pore diameter of 300 μm.

(5) Loading of TGFβ: same as Embodiment 1.

### 2. Preparation of Bone Layer: Same as Embodiment 1.

### 3. Preparation of Osteochondral Scaffold

The above TGFβ-GelMA/HAMA cartilage layer and HAP/PLA bone layer were connected with medical glue Golden Elephant, an adhesive layer was formed at the joint, the thickness of the adhesive layer was about 100 μm, and a TGFβ-GelMA/HAMA-HAP/PLA osteochondral scaffold was obtained, its structure schematic diagram is shown in FIG. 22.

In this embodiment, the GelMA/HAMA-HAP/PLA osteochondral scaffold is obtained if TGFβ is not loaded.

### Embodiment 10

### 1. Preparation of Cartilage Layer

(1) Synthesis of gelatin methacrylate (GelMA): same as Embodiment 1.

(2) Preparation of sodium alginate/gelatin composite crosslinking hydrogel composition: 0.02 g of sodium alginate (Alg), 0.05 g of GelMA and 5 mg of LAP were weighed and dissolved in 1 mL of PBS solution (pH=7.4), a hydrogel composition of 2% Alg/5% GelMA/0.5% LAP was prepared at 37° C.

(3) The hydrogel composition was poured into a prefabricated mold, photocrosslinking was realized under the irradiation of a light source with a wavelength of 405 nm and an intensity of 10 mW/cm²; the molded hydrogel was removed from the mold and soaked in 0.1 M CaCl₂ for 2 hours to achieve chemical crosslinking to obtain a composite photocrosslinked cured hydrogel; the prepared composite photocrosslinked cured hydrogel was frozen in a −20° C. refrigerator for 2 hours, and then freeze-dried in a freeze-dryer to obtain a Alg/GelMA cartilage layer.

(4) Loading of mesenchymal stem cells (MSCs): MSCs were digested with trypsin, the cells were collected by centrifugation, the cell suspension was added dropwise to the above Alg/GelMA cartilage layer, incubated for 1 hour, the culture medium was added, and cultured in a cell culture incubator at 37° C./5% CO₂ for 7 days to obtain a MSC-Alg/GelMA cartilage layer.

### 2. Preparation of Bone Layer

(1) Preparation of polycaprolactone high polymer material (TCP/PCL) loaded with tricalcium phosphate (TCP)

5 g of polycaprolactone (PCL) was weighed and dissolved in 20 mL of dichloromethane until it was completely dissolved, 1 g of tricalcium phosphate (TCP) was added, stirred until the solution was homogeneous, placed in a fume hood until dichloromethane evaporated, dried in a vacuum drying oven for 24 hours, crushed into powder with a pulverizer to obtain a TCP/PCL high polymer material.

(2) 3D printing with TCP/PCL high polymer material: The TCP/PCL high polymer material was printed by a fused deposition 3D printer (printing temperature: 140° C.; printing pressure: 40 PSI; printing speed: 50 mm/s; filling rate: 50%) to obtain a TCP/PCL bone layer. The obtained TCP/PCL bone layer was a cylinder (5 mm in diameter and 3 mm in height) with a porosity of 50% and a pore diameter of 300 μm.

### 3. Preparation of Osteochondral Scaffold

The above MSC-Alg/GelMA cartilage layer and TCP/PCL bone layer were connected with medical glue Golden Elephant, an adhesive layer was formed at the joint, the thickness of the adhesive layer was about 100 μm, and a MSC-Alg/GelMA-TCP/PCL osteochondral scaffold was obtained, its structure schematic diagram is shown in FIG. 22.

In this embodiment, the Alg/GelMA-TCP/PCL osteochondral scaffold is obtained if MSC is not loaded.

### Embodiment 11

### 1. Preparation of Cartilage Layer

(1) Synthesis of gelatin methacrylate (GelMA): same as Embodiment 1.

(2) Preparation of sodium alginate/gelatin composite crosslinked hydrogel composition: same as Embodiment 3.

(3) Preparation of Alg/GelMA cartilage layer by 3D printing demolding method:

S1, a suitable cartilage layer mold was designed according to the cartilage layer and the polyacrylate photosensitive resin was 3D printed;

S2, the hydrogel composition was poured into the cartilage layer mold, realizing in-situ photocrosslinking under the irradiation of a light source with a wavelength of 405 nm and an intensity of 10 mW/cm², and then immersed in 0.1 M CaCl₂ solution for 2 hours to realize chemical crosslinking to obtain the Alg/GelMA composite photocrosslinked cured hydrogel-cartilage layer mold complex;

S3, demolding: the cartilage layer mold in the Alg/GelMA composite photocrosslinked cured hydrogel-cartilage layer mold complex was dissolved with dichloromethane to obtain the Alg/GelMA composite photocrosslinked cured hydrogel.

S4, the prepared Alg/GelMA composite photocrosslinked cured hydrogel was frozen in a −20° C. refrigerator for 2 hours, and then freeze-dried in a freeze-dryer to obtain a Alg/GelMA cartilage layer. Wherein, the obtained Alg/GelMA cartilage layer was a cylinder (5 mm in diameter and 1 mm in height) with a porosity of 50% and a pore diameter of 250 μm.

(4) Loading of mesenchymal stem cells (MSCs): same as Embodiment 3.

### 2. Preparation of Bone Layer: same as Embodiment 3

### 3. Preparation of Osteochondral Scaffold

The above MSC-Alg/GelMA cartilage layer and TCP/PCL bone layer were connected with medical glue Golden Elephant, an adhesive layer was formed at the joint, the thickness of the adhesive layer was about 100 μm, and a MSC-Alg/GelMA-TCP/PCL osteochondral scaffold was obtained, its structure schematic diagram is shown in FIG. 22.

In this embodiment, the Alg/GelMA-TCP/PCL osteochondral scaffold is obtained if MSC is not loaded.

### Embodiment 12

### 1. Synthesis of Gelable Components

(1) Synthesis of gelatin methacrylate (GelMA): same as Embodiment 1.

(2) Synthesis of hyaluronic acid methacrylate (HAMA): same as Embodiment 1.

(3) Synthesis of chondroitin sulfate methacrylate (CSMA): chondroitin sulfate (10 g, 30 kDa) was dissolved in 100 mL of deionized water, cooled to 0-4° C., 50 mL of methacrylic anhydride was added, 50 mL of 5M NaOH aqueous solution was slowly added dropwise, reacted for 24 hours, the reaction solution was poured into a dialysis bag (mWCO 7000), dialyzed with deionized water for 2-3 days, freeze-dried to obtain the chondroitin sulfate methacrylate (9 g). According to the hydrogen nuclear magnetic resonance (¹H NMR), the degree of methacrylation of CSMA was calculated to be 40% (methacrylation degree of CSMA=peak area of methacrylate-vinyl at 5.6 ppm/peak area of N-acetyl glucosamine at 1.9 ppm*100%).

### 2. Preparation of Hydrogel Composition

(1) Hydrogel composition of the cartilage layer: 20 g of gelatin methacrylate (GelMA), 1 g of hyaluronic acid methacrylate (HAMA) and 1 g of chondroitin sulfate methacrylate (CSMA) were weighed and dissolved in deionized water at 50° C., 0.1 g of initiator LAP was added to prepare a hydrogel of the cartilage layer; wherein the percentage is the mass (g) of the components per 100 mL of gel medium;

(2) Hydrogel composition of the transition layer: 20 g of gelatin methacrylate (GelMA) and 10 g of bioactive glass were weighed and dissolved in deionized water at 50° C., 0.25 g of initiator LAP was added to prepare a hydrogel of the transition layer; wherein the percentage is the mass (g) of the components per 100 mL of gel medium;

(3) Hydrogel composition of the bone layer: 20 g of gelatin methacrylate (GelMA) and 50 g of bioactive glass were weighed and dissolved in deionized water at 50° C., 0.2 g of initiator LAP was added to prepare a hydrogel of the bone layer; wherein the percentage is the mass (g) of the components per 100 mL of gel medium;

### 3. Integration by 3D printing demolding method

S1, a suitable osteochondral scaffold mold was designed according to the osteochondral scaffold and the polyvinyl alcohol (PVA) was 3D printed;

S2, sequentially pouring the hydrogel composition of the bone layer, the adhesive layer and the cartilage layer into the osteochondral scaffold mold, in-situ photocrosslinking was realized under the irradiation of a light source with a wavelength of 405 nm and an intensity of 10 mW/cm² to obtain the photocrosslinked cured hydrogel-osteochondral scaffold mold complex;

S3, demolding: the osteochondral scaffold mold in the photocrosslinked cured hydrogel-osteochondral scaffold mold complex was dissolved with purified water to obtain a photocrosslinked cured hydrogel.

S4, the prepared photocrosslinked cured hydrogel was frozen in a −20° C. refrigerator for 2 hours, and then freeze-dried in a freeze-dryer to obtain an integrated osteochondral scaffold.

The obtained osteochondral scaffold was a cuboid (30 mm*30 mm at the bottom and 3 mm in height) with a porosity of 50% and a pore diameter of 250 μm.

### Effect Embodiment 2: Cytocompatibility Test of Osteochondral Scaffold

The GelMA/HAMA-HAP/PLA osteochondral scaffold prepared in Embodiment 8 and the Alg/GelMA-TCP/PCL osteochondral scaffold prepared in Embodiment 11 were used as examples.

The mesenchymal stem cells (MSCs) were digested with trypsin, the cells were collected by centrifugation, the cell suspension was added dropwise to the above osteochondral scaffold, incubated for 1 hour, the culture medium was added, and cultured in a cell culture incubator at a condition of 37° C./5% CO₂ for 24 hours. Before the test, the cell culture medium was aspirated and washed several times with PBS, 1 mL of live/dead cell double staining reagent (10 μM of calcein and 15 μM of ethidium dimer dissolved in 5 mL of PBS) was added, incubated with the cells at 37° C. for 30 min.

The adhesion and survival of the cells inside the osteochondral scaffold were observed using confocal fluorescence microscope. Live cells exhibited calcein staining activity and emitted green fluorescence at 433 nm; dead cells were stained by ethidium bromide and emitted red fluorescence under 543 nm excitation. As seen in FIG. 25, the osteochondral scaffold of the present disclosure has good cytocompatibility and is able to grow into the through-hole structure of the scaffold material.

### Effect Embodiment 3: Application of Osteochondral Scaffold in the Repair of Osteochondral Composite Defects in Rabbits

The GelMA/HAMA-HAP/PLA osteochondral scaffold prepared in Embodiment 8 was used as an example.

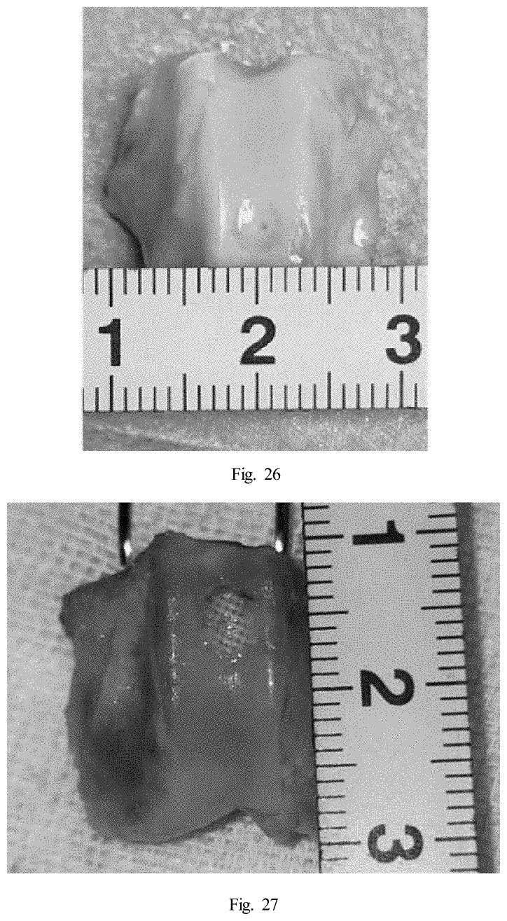

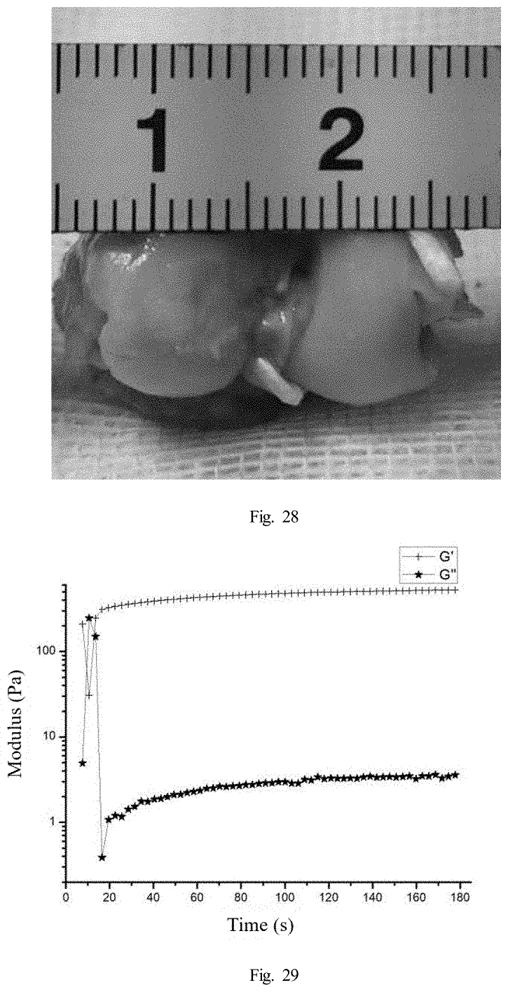

New Zealand male white rabbits were used, and each rabbit was established with a osteochondral composite defect model. Before the experiment, the rabbits were randomly divided into groups according to body weight (3 per group): a: blank control group; b: bone layer scaffold (HAP/PLA) negative control group; c: osteochondral scaffold (GelMA/HAMA-HAP/PLA) group. During the operation, the scaffold was used to fill the osteochondral defect in the rabbit joint. Twelve weeks after the operation, the rabbits in the experiment were sacrificed by intravenous air injection, and the injured joints were extracted for evaluation of the experimental repair effect. The general observation photos of the injured joints are shown in FIGS. 26-28. FIG. 26 shows the blank control group, where the new tissue is barely visible due to the absence of scaffold placement. FIG. 27 shows the negative control group with only the bone layer scaffold, where new cartilage did not grow at all and only the not yet degraded bone layer scaffold was seen due to the lack of cartilage layer. FIG. 28 shows the osteochondral scaffold group, where it can be seen that new tissue was formed at the implanted osteochondral scaffold, and had a similar appearance to the surrounding normal tissue with a better repair effect.

In the following Embodiments 13-18 and Comparative Embodiments 1-3, gelatin methacrylate (GelMA: SR-3DP-0201), hyaluronic acid methacrylate (HAMA: SR-3DP-0301), chondroitin sulfate methacrylate (CSMA: SR-3DP-0401) and LAP were purchased from SinoBioPrint (Shanghai) Biotech Ltd. Wherein, the methacrylation degree of GelMA is 65%; the molecular weight of HAMA is 900 kDa, the methacrylation degree of HAMA is 40%; the molecular weight of CSMA is 30 kDa, the methacrylation degree of CSMA is 40%.

### Embodiment 13

0.05 g of GelMA, 0.02 g of HAMA, 0.02 g of CSMA and 2.5 mg of LAP were weighed and dissolved in 1 mL of 0.9% NaCl solution (pH=7.4) at 37° C., the mixture was poured into a prefabricated mold, photocrosslinking was realized under blue light irradiation with a wavelength of 405 nm and an intensity of 10 mW/cm² to obtain a cured hydrogel of 5% GelMA/2% HAMA/2% CSMA. Then, the prepared cured hydrogel was pre-cooled in a −20° C. refrigerator for 2 hours, and then freeze-dried in a freeze-dryer for 24 hours to prepare a bionic cartilage scaffold of 5% GelMA/2% HAMA/2% CSMA.

### Embodiment 14

0.1 g of GelMA, 0.01 g of HAMA, 0.03 g of CSMA and 2.5 mg of LAP were weighed and dissolved in 1 mL of PBS solution (pH=7.4) at 37° C., the mixture was poured into a prefabricated mold, photocrosslinking was realized under blue light irradiation with a wavelength of 405 nm and an intensity of 10 mW/cm² to obtain a cured hydrogel of 10% GelMA/1% HAMA/3% CSMA. Then, the prepared cured hydrogel was pre-cooled in a −20° C. refrigerator for 2 hours, and then freeze-dried in a freeze-dryer for 20 hours to prepare a bionic cartilage scaffold of 10% GelMA/1% HAMA/3% CSMA.

### Embodiment 15

0.15 g of GelMA, 0.01 g of HAMA, 0.01 g of CSMA and 2.5 mg of LAP were weighed and dissolved in 1 mL of PBS solution (pH=7.4) at 37° C., the mixture was placed in a blue light assisted extrusion 3D printer, and 3D printed under blue light irradiation with a wavelength of 405 nm and an intensity of 10 mW/cm² to obtain a cured hydrogel of 15% GelMA/1% HAMA/1% CSMA. Then, the prepared cured hydrogel was pre-cooled in a −20° C. refrigerator for 2 hours, and then freeze-dried in a freeze-dryer for 12 hours to prepare a bionic cartilage scaffold of 15% GelMA/1% HAMA/1% CSMA.

### Embodiment 16

Preparation of bionic cartilage scaffold by 3D printing demolding method:

(1) a suitable mold was designed according to the desired bionic cartilage scaffold, and the mold was 3D printed using PLA as the sacrificial material;

(2) 0.08 g of GelMA, 0.01 g of HAMA, 0.03 g of CSMA and 2.5 mg of LAP were weighed and dissolved in the culture medium at 37° C., the mixture was poured into the mold, photocrosslinking was realized under blue light irradiation with a wavelength of 405 nm and an intensity of 10 mW/cm² to obtain a cured hydrogel-mold complex of 8% GelMA/1% HAMA/3% CSMA;

(3) the cured hydrogel-mold complex was immersed in dichloromethane, which dissolved the mold to obtain an cured hydrogel of 8% GelMA/1% HAMA/3% CSMA;

(4) the cured hydrogel was pre-cooled in a −20° C. refrigerator for 2 hours, and then freeze-dried in a freeze-dryer for 8 hours to prepare a bionic cartilage scaffold of 8% GelMA/1% HAMA/5% CSMA.

### Embodiment 17

0.08 g of GelMA, 0.01 g of HAMA, 0.03 g of CSMA, 0.02 g of sodium alginate (Alg) and 2.5 mg of LAP were weighed and dissolved in 1 mL of culture medium at 37° C., the mixture was placed in a blue light assisted extrusion 3D printer, and 3D printed under blue light irradiation with a wavelength of 405 nm and an intensity of 10 mW/cm² to obtain a cured hydrogel of 8% GelMA/1% HAMA/3% CSMA/2% Alg. Then, the obtained cured hydrogel was pre-cooled in a −20° C. refrigerator for 2 hours, and then freeze-dried in a freeze-dryer for 20 hours to prepare a bionic cartilage scaffold of 8% GelMA/1% HAMA/3% CSMA/2% Alg.

### Embodiment 18

0.08 g of GelMA, 0.01 g of HAMA, 0.03 g of CSMA, 0.1 g of PEGDA and 0.01 g of LAP were weighed and dissolved in 1 mL of culture medium at 37° C., the mixture was poured into a prefabricated silicone mold, photocrosslinking was realized under blue light irradiation with a wavelength of 405 nm and an intensity of 10 mW/cm² to obtain a cured hydrogel of 8% GelMA/1% HAMA/3% CSMA/10% PEGDA; Then, the prepared cured hydrogel was pre-cooled in a −20° C. refrigerator for 2 hours, and then freeze-dried in a freeze-dryer for 20 hours to prepare a bionic cartilage scaffold of 8% GelMA/1% HAMA/3% CSMA/10% PEGDA.

### Comparative Embodiment 1

0.05 g of GelMA, 0.02 g of HAMA, 0.32 g of CSMA and 2.5 mg of LAP were weighed and dissolved in 1 mL of 0.9% NaCl solution (pH=7.4) at 37° C., finding that excessive CSMA content would affect the mechanical properties of the hydrogel.

### Comparative Embodiment 2

0.55 g of GelMA, 0.02 g of HAMA, 0.02 g of CSMA and 2.5 mg of LAP were weighed and dissolved in 1 mL of 0.9% NaCl solution (pH=7.4) at 37° C., finding that GelMA was difficult to dissolve completely and could not continue to prepare the hydrogel.

### Comparative Embodiment 3

0.05 g of GelMA, 0.35 g of HAMA, 0.02 g of CSMA and 2.5 mg of LAP were weighed and dissolved in 1 mL of 0.9% NaCl solution (pH=7.4) at 37° C., finding that HAMA was difficult to dissolve completely and could not continue to prepare the hydrogel.

### Effect Embodiment 4: Rheological Analysis of Hydrogel

0.05 g of GelMA, 0.02 g of HAMA, 0.02 g of CSMA and 2.5 mg of LAP were weighed and dissolved in 1 mL of 0.9% NaCl solution (pH=7.4) at 37° C. Dynamic rheological experiment was performed on a Haake Mars rotational rheometer using a parallel plate (P20 TiL, 20 mm in diameter) at 25° C. A 60 s scan test was performed under blue light (405 nm, 30 mW/cm²) irradiation with 10% strain (CD mode), 1 Hz frequency and 0.5 mm gap. The modulus variation with time is shown in FIG. 29, the gel point is the time when the storage modulus (G′) exceeds the loss modulus (G″), and it can be seen that the gel point is 3 s. The storage modulus reached its maximum at about 10 s, indicating a very rapid curing, and the value of the storage modulus was maintained until the end of the test, indicating that the formed gel structure was stable.

### Effect Embodiment 5: Mechanical Properties of Hydrogel

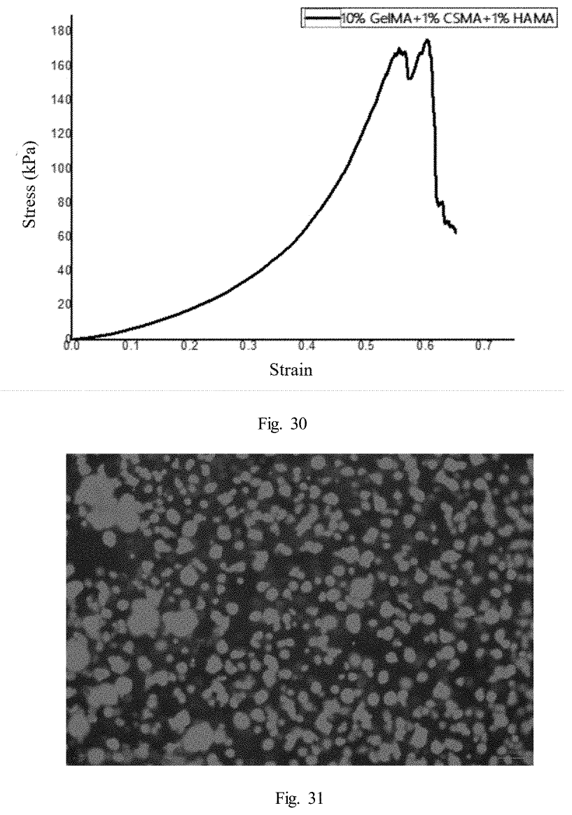

A solution of 5% GelMA+1% HAMA+1% CSMA+0.25% LAP was prepared and placed into a cylindrical mold with a diameter of 10 mm and a height of 8 mm, photocrosslinking was realized under blue light irradiation with a wavelength of 405 nm to obtain a curing hydrogel scaffold of 5% GelMA+1% HAMA+1% CSMA as the test sample. The mechanical properties of the cured hydrogel scaffold were tested with a GT-TCS-2000 single column instrument, the compression speed was set to 1 mm/min, crushing stopped, and the stress-strain curve was obtained, as shown in FIG. 30. The maximum pressure of the cured hydrogel scaffold before breaking is the ultimate stress, and the elastic modulus was calculated based on the slope of the stress-strain curve of 15%-20%. It can be seen from FIG. 30 that the ultimate stress of the cured hydrogel scaffold was 170.8 kPa, and the elastic modulus was 123.7 kPa.

### Effect Embodiment 6: Cytocompatibility Test of Bionic Cartilage Scaffold

0.05 g of GelMA, 0.02 g of HAMA and 0.02 g of CSMA were weighed and mixed with a culture medium containing P2 generation rabbit chondrocytes, 2.5 mg LAP was added, the mixture was cured into a gel for 5 seconds under blue light irradiation with a wavelength of 405 nm, after 7 days of culture, cell survival was detected using Calcein/AM live/dead cell staining kit, and the results are shown in FIG. 31, with the cell survival in the hydrogel>90%.

### Effect Embodiment 7: Osteochondral Defect Repair Rest

### 1. Preparation of Osteochondral Scaffold

(1) A bionic cartilage scaffold of 5% GelMA/2% HAMA/2% CSMA was prepared according to the method in Embodiment 13.

(2) 10 g of polylactic acid (PLA) was weighed and dissolved in 20 mL of dichloromethane until it was completely dissolved, 1 g of hydroxyapatite (HAP) was added, stirred until the solution was homogeneous, placed in a fume hood until dichloromethane evaporated, and dried in a vacuum drying oven for 24 hours to obtain a HAP/PLA high polymer material. The HAP/PLA high polymer material was printed by a fused deposition 3D printer (print head temperature: 210° C.; platform temperature: 50° C.; printing speed: 60 mm/s; filling rate: 50%) to obtain a HAP/PLA bone layer.

(3) The bionic cartilage scaffold and the HAP/PLA bone layer were connected with medical glue (Golden Elephant) to obtain an osteochondral scaffold.

### 2. Osteochondral Defect Repair Test

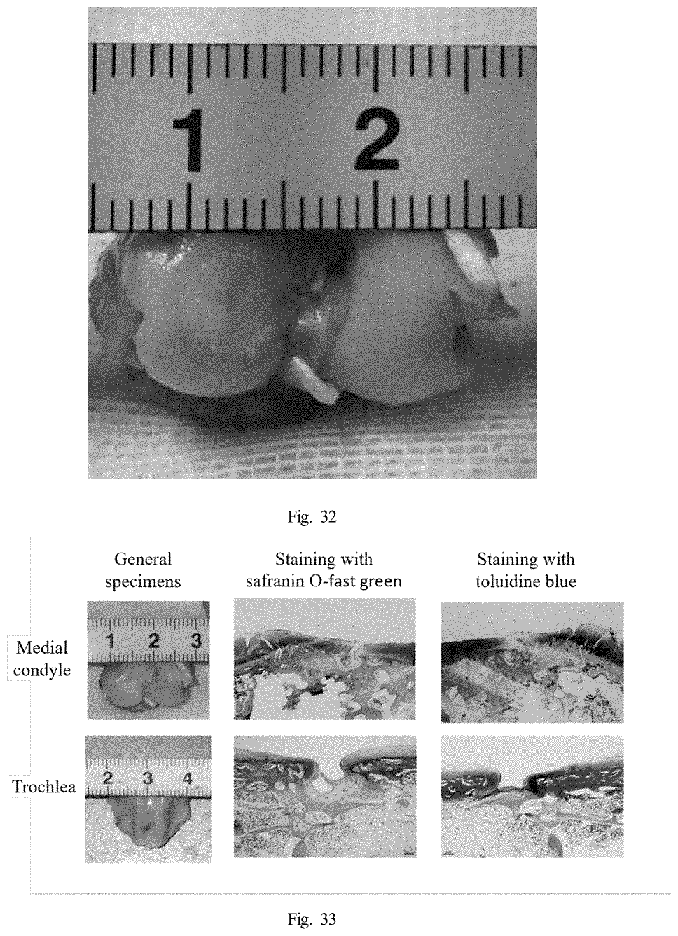

Fifteen New Zealand white rabbits, female, weighing 3-3.5 kg, 5 months old and skeletally mature, were drilled at the trochlea and medial condyle of the femur to create an osteochondral defect model. The above osteochondral scaffold was implanted in the medial condyle defect as the experimental group; GelMA-based cartilage hydrogel was filled around the cartilage layer, and the trochlea was left untreated as the blank control group. Euthanasia was performed after 12 weeks for general observation (see FIG. 32) and histological examination (see FIG. 33). As shown in FIG. 32, 3 months after operation at the medial condyle where the osteochondral scaffold was implanted, smooth new cartilage could be seen and well connected and integrated with the surrounding normal cartilage. As shown in FIG. 33, the cartilage defect at the trochlea of the blank control group was severe, and it was still an empty pit. The histological analysis in FIG. 33 also showed that chondrocyte lacunas were generated at the medial condyle defect, whereas no chondrocyte lacunas were generated at the trochlea of the blank control group; the red color of the cartilage matrix on staining with safranin O and the purple color on staining with toluidine blue indicated that new cartilage matrix was secreted at the medial condyle defect and cancellous bone generation was observed under the cartilage; whereas no cartilage matrix was secreted at the trochlea of the blank control group and no cancellous bone generation was observed.

Although the specific embodiments of the present disclosure have been described above, it should be understood by those skilled in the art that these are merely illustrative examples and that a variety of changes or modifications can be made to these embodiments without departing from the principles and substance of the present disclosure. Therefore, the scope of protection of the present disclosure is limited by the appended claims.

1-5. (canceled)

6. An osteochondral scaffold, comprising a cartilage layer, an adhesive layer and a bone layer, the adhesive layer being connected to the cartilage layer and the bone layer on each side; the cartilage layer, the adhesive layer and the bone layer being porous; the pores of the cartilage layer, the pores of the adhesive layer and the pores of the bone layer are communicated; the pores of the cartilage layer, the pores of the adhesive layer and the pores of the bone layer are completely or imcompletely aligned; the adhesive layer does not cover or partially covers the pores of the bone layer and the cartilage.

7. The osteochondral scaffold according to claim 6, wherein, the distribution of the pores of the cartilage layer and the bone layer is arranged vertically and crosswise.

8. A preparation method for the osteochondral scaffold according to claim 6, comprising the following steps of: connecting a bone layer and a cartilage layer, with an adhesive layer formed at the joint; the cartilage layer, the adhesive layer and the bone layer are porous; the pores of the cartilage layer, the pores of the adhesive layer and the pores of the bone layer are communicated; the pores of the cartilage layer, the pores of the adhesive layer and the pores of the bone layer are completely or imcompletely aligned; the adhesive layer does not cover or partially covers the pores of the bone layer and the cartilage.

9. The preparation method for the osteochondral scaffold according to claim 8, wherein, the cartilage layer is prepared by crosslinking curing with a hydrogel composition as a raw material; wherein, the hydrogel composition comprises at least a gelable component and a gel medium;
the gelable component comprises a natural gelable component and a synthetic gelable component; wherein, the natural gelable component comprises one or more selected from a group consisting of natural proteins, natural protein modification products, natural protein degradation products, modification products of natural protein degradation products, natural polysaccharides, natural polysaccharide modification products, natural polysaccharide degradation products and modification products of natural polysaccharide degradation products;
the synthetic gelable component comprises one or more selected from a group consisting of two-arm or multi-arm polyethylene glycol diacrylate, polyethyleneimine, synthetic polypeptide, polyacrylic acid, polymethacrylic acid, polyacrylate, polymethacrylate, polyacrylamide, polymethacrylamide, polyvinyl alcohol and polyvinylpyrrolidone;
the gel medium is one or more selected from a group consisting of purified water, saline, cell culture medium, calcium salt solution and phosphate buffered solution;
the method of the crosslinking curing comprises one or more selected from a group consisting of physical crosslinking, chemical crosslinking, enzymatic crosslinking and photocrosslinking.

10. The preparation method for the osteochondral scaffold according to claim 8, wherein, the preparation method for the bone layer is 3D printing with the material of the bone layer; the 3D printing is carried out using a fused deposition 3D printer; the material of the bone layer is polylactic acid, polylactic-co-glycolic acid or polycaprolactone.

11. The preparation method for the osteochondral scaffold according to claim 8, wherein, the osteochondral scaffold is prepared by a 3D printing demolding method, comprising the following steps of:
S1, carrying out 3D printing with a sacrificial material to obtain an osteochondral scaffold mold; wherein the sacrificial material is a hard high polymer material which can be dissolved in a solvent;
S2, sequentially pouring the hydrogel compositions of the bone layer, the adhesive layer and the cartilage layer into the osteochondral scaffold mold for crosslinking to obtain a cured hydrogel-osteochondral scaffold mold complex;
S3, demolding: dissolving the osteochondral scaffold mold with a solvent to obtain a cured hydrogel;
S4, freeze-drying the obtained cured hydrogel to obtain the osteochondral scaffold.

12. An application of the osteochondral scaffold according to claim 6 in the repair of osteochondral defects to the subject.

13-20. (canceled)

21. The osteochondral scaffold according to claim 6, wherein, the height ratio of the bone layer and the cartilage layer is 1:(0.1-1).

22. The osteochondral scaffold according to claim 6, wherein, the porosity of the cartilage layer and the bone layer is 20%-70%, the porosity of the osteochondral scaffold is 20%-70%.

23. The osteochondral scaffold according to claim 6, wherein, the pore diameter of the pores of the bone layer is equal to that of the pores of the cartilage layer.

24. The osteochondral scaffold according to claim 6, wherein, the osteochondral scaffold is a cylinder; the cylinder has a diameter of 2-20 mm, the cylinder has a height of 2-10 mm, the height of the adhesive layer is from 5 μm to 2 mm;
or, the osteochondral scaffold is a cuboid, the bottom surface of the cuboid is a square, the square has a side length of 2-30 mm, the cuboid has a height of 2-10 mm, the height of the adhesive layer is from 5 μm to 2 mm.

25. The osteochondral scaffold according to claim 6, wherein, the material of the cartilage layer is a hydrogel material;
the material of the bone layer is polylactic acid, polylactic-co-glycolic acid or polycaprolactone;
the adhesive layer is formed by a medical glue; or, the material of the adhesive layer is a hydrogel material.

26. The preparation method for the osteochondral scaffold according to claim 9, wherein, the hydrogel composition of the cartilage layer comprises the following components in parts by mass: 1-50 parts of gelatin methacrylate, 0-30 parts of hyaluronic acid methacrylate, 0-30 parts of chondroitin sulfate methacrylate, 0.01-1 part of photoinitiator and gel.

27. The preparation method for the osteochondral scaffold according to claim 25, wherein, the amount of the gel medium is such that in the hydrogel composition: 5%-30% of gelatin methacrylate, 0.5%-2% of hyaluronic acid methacrylate, 0.1%-5% of chondroitin sulfate methacrylate, 0.01%-1% of photoinitiator; wherein the percentage is the mass (g) of the components per 100 mL of gel medium.

28. The preparation method for the osteochondral scaffold according to claim 8, wherein, the preparation method for the cartilage layer is a perfusion method, comprising the following steps: pouring the hydrogel composition of the cartilage layer into a cartilage mold for photocrosslinking to obtain a cured hydrogel; freeze-drying the cured hydrogel to obtain the cartilage layer;
or, the preparation method for the cartilage layer is a direct 3D printing method, comprising the following steps: 3D printing the hydrogel composition of the cartilage layer and performing photocrosslinking at the same time to obtain a cured hydrogel; freeze-drying the cured hydrogel to obtain the cartilage layer.

29. The preparation method for the osteochondral scaffold according to claim 8, wherein, the preparation method for the cartilage layer is a 3D printing demolding method, comprising the following steps:
S1, carrying out 3D printing with a sacrificial material to obtain a cartilage layer mold; wherein the sacrificial material is a hard high polymer material which can be dissolved in a solvent;
S2, pouring the hydrogel composition of the cartilage layer into the cartilage layer mold for crosslinking to obtain a cured hydrogel-cartilage layer mold complex;
S3, demolding: dissolving the cartilage layer mold with a solvent to obtain a cured hydrogel;
S4, freeze-drying the obtained cured hydrogel to obtain the cartilage layer.

30. The preparation method for the osteochondral scaffold according to claim 8, wherein, the preparation method of the cartilage layer further comprises a step of loading a cartilage promoting component.

31. The preparation method for the osteochondral scaffold according to claim 8, wherein, the bone layer is prepared by crosslinking curing with a hydrogel composition as a raw material;
the hydrogel composition for the bone layer comprises the following components: 5%-30% of gelatin methacrylate, 2.5%-50% of bioactive glass and 0.01%-1% of photoinitiator; wherein the percentage is the mass (g) of the components per 100 mL of gel medium.

32. The preparation method for the osteochondral scaffold according to claim 8, wherein, the preparation method for the bone layer further comprises a step of loading a bone promoting component;
the method for loading the bone promoting component comprises: grinding the material of the bone layer into powder and then mixing it with the bone promoting component; or, dissolving the material of the bone layer in a solvent and then mixing it with the bone promoting component, and then allowing the solvent to evaporate.

33. The preparation method for the osteochondral scaffold according to claim 11, wherein, the hydrogel composition of the bone layer comprises the following components: 5%-30% of gelatin methacrylate, 2.5%-50% of bioactive glass and 0.01%-1% of photoinitiator; the hydrogel composition of the adhesive layer comprises the following components: 5%-30% of gelatin methacrylate, 10%-20% of bioactive glass and 0.01%-1% of photoinitiator; the hydrogel composition of the cartilage layer comprises the following components: 5%-30% of gelatin methacrylate, 0.5%-2% of hyaluronic acid methacrylate, 0.5%-5% of chondroitin sulfate methacrylate, and 0.01%-1% of photoinitiator; wherein the percentage is the mass (g) of the components per 100 mL of gel medium.
